(19) United States
(12) Patent Application Publication
(10) Pub. No.: US 2026/0130906 A1
(43) Pub. Date: May 14, 2026

(54) THERAPY COMPRISING ANTI-CD19 ANTIBODY AND SUMO-ACTIVATING ENZYME INHIBITOR

(71) Applicants: TAKEDA PHARMACEUTICAL COMPANY LIMITED, Osaka (JP); INCYTE CORPORATION, Wilmington, DE (US); MORPHOSYS AG, Planegg (DE)

(72) Inventors: Dennis HUSZAR, Cambridge, MA (US); Igor PROSCURSHIM, North Andover, MA (US)

(21) Appl. No.: 18/705,192

(22) PCT Filed: Oct. 28, 2022
(86) PCT No.: PCT/IB2022/060413

§ 371 Date: Apr. 26, 2024

Related U.S. Application Data
(60) Provisional application No. 63/273,397, filed on Oct. 29, 2021.

(51) Int. Cl. A61K 31/506; A61K 39/00; A61P 35/00; C07K 16/28

(52) U.S. Cl. CPC A61K 31/506; A61P 35/00; C07K 16/2803; A61K 2039/505; C07K 2317/52; C07K 2317/565; C07K 2317/72; C07K 2317/732

(57) ABSTRACT

The present disclosure provides methods, pharmaceutical compositions, and kits for treating cancer in patients in need thereof. The methods comprise administering to a patient in need a small ubiquitin-like modifier (SUMO) activating enzyme (SAE) inhibitor, such as [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxy-cyclopentyl]methyl sulfamate (Compound I-263a) or a pharmaceutically acceptable salt, in combination with one or more anti-CD19 antibodies. Also provided are medicaments for use in treating cancer.

### FIELD

The present disclosure is directed to a combination comprising an anti-CD19 antibody thereof and a small ubiquitin-like modifier (SUMO) activating enzyme (SAE) inhibitor for use in the treatment of various cancers.

### BACKGROUND

Non-Hodgkin lymphoma (NHL) is among the most common cancers in the United States and Europe with more than 70,000 and 93,000 new cases diagnosed every year, respectively. Siegel R. L., et al., CA Cancer J. Clin. 68(1): 7-30 (2018); Ferlay J., et al., Eur. J. Cancer 103:356-87 (2018). NHL is a heterogeneous group of malignancies with varying clinical characteristics that are optimally managed through a range of different treatment modalities. The spectrum of NHL includes more indolent variants such as follicular and marginal zone lymphomas, to more aggressive subtypes such as diffuse large B-cell lymphoma (DLBCL) and Burkitt lymphoma (BL). While systemic chemotherapy is a mainstay of treatment for most NHL variants, antitumor directed monoclonal antibodies have an important role in the treatment of this disease. Oflazoglu E., et al., MAbs 2(1): 14-9 (2010). Monoclonal antibodies such as rituximab, which targets the B-cell antigen CD20, are part of the standard treatment regimens for many B-cell NHLs. Keating G M, Drugs 70(11): 1445-76 (2010). However, once NHL becomes refractory to standard chemotherapy and antibody-based therapies, the overall prognosis is poor, with limited long-term survival. Thus, novel and effective therapies are needed to address this high unmet medical need.

Indolent NHL (iNHL) represents 40% of all NHL subtypes, with follicular lymphoma occurring with the greatest frequency. Harris N. L., et al., Ann. Oncol. 10(12): 1419-32 (1999). iNHL presents with a broad spectrum of disease characteristics. Patients often experience a chronic relapsing and remitting disease course and are exposed to several successive treatment regimens, resulting eventually in death due to disease progression. In general, treatment is reserved for patients who develop significant symptoms or who are sufficiently high risk to merit early therapy. Gribben J. G., Blood 109(11): 4617-26 (2007).

For patients with iNHL who initially respond (complete or partial response (PR) with a time to progression of at least 6 months) and then experience relapse after single-agent rituximab, retreatment with either rituximab alone or in combination with chemotherapy is frequently given. Gribben J. G., Blood 109(11): 4617-26 (2007); Kahl B. S., et al., J. Clin. Oncol. 32(28): 3096-102 (2014); NCCN Clinical Practice Guidelines in Oncology (NCCN Guidelines): B-Cell Lymphomas (Version 3.2019), National Comprehensive Cancer Network, May 6, 2019. Patients who become refractory to rituximab alone or in combination with chemotherapy have limited options for effective treatment.

Aggressive Non-Hodgkin Lymphoma (aNHL) accounts for approximately 30 to 40% of all NHL (Project TN-HsLC, Blood 89(11): 3909-18 (1997)) and DLBCL is the most common histological subtype. Beham-Schmid C., Aggressive lymphoma 2016: revision of the WHO classification, Memo 10(4): 248-54 (2017). Combination chemotherapy with the addition of rituximab is standard of care for patients with newly diagnosed DLBCL. However, approximately 40% of patients with DLBCL relapse following initial immunochemotherapy. Vaidya R., et al., Ann. Oncol. 25(11): 2124-33 (2014). For eligible patients, salvage chemotherapy regimens followed by autologous stem cell transplantation is the standard of care. However, many patients are not eligible for transplantation due to age and other medical co-morbidities. While various salvage regimens comprising combination chemotherapy are available for relapsed/refractory disease, no standard salvage regimen exists currently. There remains an unmet need for NHL patients who have early relapses or who are not responsive to anti-CD20 treatment regimens.

Small ubiquitin-like modifier (SUMO) activating enzyme (SAE) inhibitors are examples of small molecules that can be used for targeted therapies. SUMO is a member of the ubiquitin-like protein (Ubl) family that covalently conjugate to cellular proteins in a manner similar to Ub-conjugation (Kerscher, O. et al., Annu Rev Cell Dev Biol. 22:159-80 (2006)). Mammalian cells express three major isoforms: SUMO1, SUMO2, and SUMO3. SUMO2 and SUMO3 share ˜95% amino acid sequence homology but have ˜45% sequence homology with SUMO1 (Kamitani, T., et al., J Biol Chem. 273(18): 11349-53 (1998)). SUMO proteins can conjugate to a single lysine residue of a protein (monosumoylation) or to a second SUMO protein that is already conjugated to a protein forming a SUMO chain (polysumoylation). Only SUMO2/3 can form such chains because they possess internal consensus SUMO modification sites (Tatham, M. H., et al., J Biol Chem. 276(38): 35368-74 (2001)). An additional isoform, SUMO4, is found in kidney, lymph node and spleen cells, but it is not known whether SUMO4 can conjugate to cellular proteins.

SUMO1, SUMO2 and SUMO3 are activated in an ATP-dependent manner by SAE (see, for example, U.S. Patent Application Publication No. 2010/0160177 A1 (FIG. 1B) and U.S. Pat. No. 9,434,765 B2 (FIG. 2)). SAE is a heterodimer that consists of SAE1 (SUMO-activating enzyme subunit 1) and SAE2 (UBA2). SAE, like other E1 activating enzymes, uses ATP to adenylate the C-terminal glycine residue of SUMO. In a second step, a thioester intermediate is then formed between the C-terminal glycine of SUMO and a cysteine residue in SAE2. Next, SUMO is transferred from the E1 to the cysteine residue of the SUMO conjugating enzyme (E2), UBC9. Unlike the Ub pathway that contains many E2 enzymes, Ubc9 is currently the only known conjugating enzyme for SUMO and functions with SUMO1, SUMO2, and SUMO3 proteins. SUMO proteins then conjugate to the target protein, either directly or in conjunction with an E3 ligase, through isopeptide bond formation with the epsilon amino group of a lysine side chain on a target protein. Several SUMO E3 ligases, including PIAS (protein inhibitor of activated signal transducer and activator of transcription protein) proteins and Ran-binding protein 2 (RanBP2), and polycomb 2 (Pc2), have been identified (Johnson, E. S., and Gupta, A. A, Cell. 106(6): 735-44 (2001); Pichler, A., et al., Cell. 108(1): 109-20 (2002); Kagey, M. H., et al., Cell. 113(1): 127-37 (2003)). Once attached to cellular targets, SUMO modulates the function, subcellular localization, complex formation and/or stability of substrate proteins (Müller, S., et al., Nat Rev Mol Cell Biol. 2(3): 202-10 (2001)). SUMO-conjugation is reversible through the action of de-sumoylating enzymes called SENPs (Hay, R. T., Trends Cell Biol. 17(8): 370-6 (2007)) and the SUMO proteins can then participate in additional conjugation cycles.

SAE-initiated SUMO-conjugation plays a major role in regulating diverse cellular processes, including cell cycle regulation, transcriptional regulation, cellular protein targeting, maintenance of genome integrity, chromosome segregation, and protein stability (Hay, R. T., Mol Cell. 18(1): 1-12 (2005); Gill, G., Genes Dev. 18(17): 2046-59 (2004)). For example, SUMO-conjugation causes changes in the subcellular localization of RanGAP1 by targeting it to the nuclear pore complex (Mahajan, R., et al., Cell. 88(1): 97-1070 (1997)). Sumoylation counteracts ubiquitination and subsequently blocks the degradation of IκB, thereby negatively regulating NF-κB activation (Desterro, J. M., et al., Mol Cell. (2): 233-9 (1998)). Sumoylation has been reported to play an important role in transcription exhibiting both repressive and stimulatory effects. Many of the transcriptional nodes that are modulated play important roles in cancer. For example, sumoylation stimulates the transcriptional activities of transcription factors such as p53 and HSF2 (Rodriguez, M. S., et al., EMBO J. 18(22): 6455-61 (1999); Goodson, M. L., et al., J Biol Chem. 276(21): 18513-8 (2001)). In contrast, SUMO-conjugation represses the transcriptional activities of transcription factors such as LEF (Sachdev, S., et al., Genes Dev. 15(23): 3088-103 (2001)) and c-Myb (Bies, J., et al., J Biol Chem. 277(11): 8999-9009 (2002)). SUMOylation has also been shown to regulate the production of Type I interferons (Crowl, J. T. and Stetson, D. B. PNAS 115(26): 6798-6803 (2018); Decque, A., et al., Nature Immunology 17(2): 140-149 (2016)). Thus, SUMO-conjugation controls gene expression and growth control pathways that are important for cancer cell survival.

Altered expression of SAE pathway components have been noted in a variety of cancer types: (Moschos, S. J., et al., Hum Pathol. 41(9): 1286-980 (2010)); including multiple myeloma (Driscoll, J. J., et al., Blood. 115(14): 2827-34 (2010)); and breast cancer (Chen, S. F., et al., Chin J Cancer. 30(9): 638-44 (2011)). In addition, preclinical studies indicate that Myc-driven cancers may be especially sensitive to SAE inhibition (Kessler, J. D., et al., Science. 335(6066): 348-53 (2012); Hoellein, A., et al., Blood. 124(13): 2081-90 (2014)). Since SUMO-conjugation regulates essential cellular functions that contribute to the growth and survival of tumor cells, targeting SAE could represent an approach to treat proliferative disorders such as cancer. (He, X., et al., Nature Chemical Biology. 13:1164-1171 (2017)). Thus, some cancers may be SAE-mediated disorders.

SAE inhibitors may also be applicable for the treatment of other diseases and conditions outside of oncology. For example, SUMO modifies proteins that play important roles in neurodegenerative diseases (Steffan, J. S., et al., Science. 304(5667): 100-4 (2004); Dorval, V., and Fraser, P. E., J Biol Chem. 281(15): 9919-24 (2006); Ballatore, C., et al., Nat Rev Neurosci. 8(9): 663-72 (2007)). Sumoylation also has been reported to play an important role in pathogenic viral infection, inflammation and cardiac function (Lee, H. R., et al., J Virol. 78(12): 6527-42 (2004); Liu, B., and Shuai, K., Mol Cell. 35(6): 731-2 (2009); Wang, J., and Schwartz, R. J., Circ Res. 107(1): 19-29 (2010)).

CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signaling and is important for B cell activation and proliferation and in the development of humoral immune responses. It acts as a co-stimulatory molecule in conjunction with CD21 and CD81 and is critical for B cell responses to T-cell-dependent antigens. The cytoplasmic tail of CD19 is physically associated with a family of tyrosine kinases that trigger downstream signaling pathways via the src-family of protein tyrosine kinases. CD19 is an attractive target for cancers of lymphoid origin since it is highly expressed in nearly all-chronic lymphocytic leukemia (CLL) and non-Hodgkin's lymphomas (NHL), as well as many other different types of leukemias, including acute lymphocytic leukemia (ALL) and hairy cell leukemia (HCL).

Tafasitamab (former names: MOR208 and XmAb®5574) is a humanized monoclonal antibody that targets the antigen CD19, a transmembrane protein involved in B-cell receptor signaling. Tafasitamab has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. Tafasitamab has or is currently being studied in several clinical trials, such as in CLL, ALL and NHL. Tafasitamab received accelerated approval from the US Food and Drug Administration (FDA) in July 2020 for use in combination with lenalidomide to treat adults with R/R DLBCL. Despite recent discoveries and developments of several anti-cancer agents, due to poor prognosis for many types of cancers including CD19-expressing tumors, there is still a need for an improved method or therapeutic approach for treating such types of cancers.

New combinations of therapeutic agents that provide a beneficial effect in the treatment of cancers are desirable in order to prolong patient's lives while maintaining a high quality of life. New combinations may provide an increased benefit as compared to each of the agents alone. In particular, combined treatment regimens may be helpful for patients suffering from disease conditions including proliferative disorders, and could potentially even decrease the rate of relapse or overcome the resistance to a particular anticancer agent sometimes seen in these patients. This is especially true in the case where the cancers may be resistant or refractory to currently available therapeutic regimens.

Thus, there is a need for new cancer treatment regimens, including combination therapies. The present inventors have confirmed that combined administration of an antibody or antibody fragment specific for CD19 together with an SAE inhibitor has superior effects on the treatment of malignant lymphomas of B cell origin, and have completed the present invention.

### SUMMARY

The present disclosure provides a novel combination for use in the treatment of a cancer, comprising an anti-CD19 antibody and an SAE inhibitor.

In one aspect, the present disclosure relates to methods of treating a cancer comprising administering to a human subject in need of said treatment an SAE inhibitor or a pharmaceutically acceptable salt thereof and an anti-CD19 antibody in combination.

In one aspect, the present disclosure relates to methods of treating a cancer comprising administering to a patient in need of said treating a combination of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD19 antibody or antibody fragment. Compound I-263a is also sometimes referred to herein as TAK-981.

In some embodiments, the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In some embodiments, the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO:
3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

In some embodiments, the anti-CD19 antibody comprises a heavy chain variable region of EVQLVESGGGLVKPGGSLKLSCAASGYTFTSYVMHWVRQAPGKGLEWIGYINPY NDGTKYNEKFQGRVTISSDKSISTAYMELSSLRSEDTAMYYCARGTYYYGTRVF DYWGQGTLVTVSS (SEQ ID NO: 7) and a light chain variable region of DIVMTQSPATLSLSPGERATLSCRSSKSLQNVNGNTYLYWFQQKPGQSPQLLIYR MSNLNSGVPDRFSGSGSGTEFTLTISSLEPEDFAVYYCMQHLEYPITFGAGTKLEI K (SEQ ID NO: 8).

In some embodiments, the anti-CD19 antibody has effector function. In another aspect the antibody or antibody fragment specific for CD19 has an enhanced effector function. In one embodiment the effector function is ADCC. In one embodiment the antibody or antibody fragment specific for CD19 has an enhanced ADCC activity. In a further embodiment the antibody or antibody fragment specific for CD19 comprises an Fc domain comprising an amino acid substitution at position S239 and/or 1332, wherein the numbering is according to the EU index as in Kabat. In a further embodiment the antibody or antibody fragment specific for CD19 comprises an Fc domain comprising an S239D amino acid substitution and an 1332E amino acid substitution, wherein the numbering is according to the EU index as in Kabat.

In some embodiments, the anti-CD19 antibody comprises a heavy chain constant region of ASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCP PCPAPELLGGPDVFLFPPKPKDTLMISRTPEVTCVVVDVSHEDPEVQFNWYVDGV EVHNAKTKPREEQFNSTFRVVSVLTVVHQDWLNGKEYKCKVSNKALPAPEEKTI SKTKGQPREPQVYTLPPSREEMTKNQVSLTCL VKGFYPSDIA VEWESNGQPENNY KTTPPMLDSDGSFFLYSKLTVDKSRWQQGNVFSCSVMHEALHNHYTQKSLSLSP GK (SEQ ID NO: 9).

In some embodiments, the anti-CD19 antibody comprises a light chain constant region of RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 10).

In some embodiments, the anti-CD19 antibody comprises a heavy chain constant region of ASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCP PCPAPELLGGPDVFLFPPKPKDTLMISRTPEVTCVVVDVSHEDPEVQFNWYVDGV EVHNAKTKPREEQFNSTFRVVSVLTVVHQDWLNGKEYKCKVSNKALPAPEEKTI SKTKGQPREPQVYTLPPSREEMTKNQVSLTCLVKGFYPSDIA VEWESNGQPENNY KTTPPMLDSDGSFFLYSKLTVDKSRWQQGNVFSCSVMHEALHNHYTQKSLSLSP GK (SEQ ID NO: 9) and a light chain constant region of RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 10).

In some embodiments, the anti-CD19 antibody comprises a heavy chain region of EVOLVESGGGLVKPGGSLKLSCAASGYTFTSYVMHWVRQAPGKGLEWIGYINPY NDGTKYNEKFQGRVTISSDKSISTAYMELSSLRSEDTAMYYCARGTYYYGTRVF DYWGQGTLVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK KVEPKSCDKTHTCPPCPAPELLGGPDVFLFPPKPKDTLMISRTPEVTCVVVDVSHE DPEVQFNWYVDGVEVHNAKTKPREEQFNSTFRVVSVLTVVHQDWLNGKEYKC KVSNKALPAPEEKTISKTKGQPREPQVYTLPPSREEMTKNQVSLTCLVKGFYPSDI AVEWESNGQPENNYKTTPPMLDSDGSFFLYSKLTVDKSRWQQGNVFSCSVMHE ALHNHYTQKSLSLSPGK (SEQ ID NO: 11) and a light chain region of DIVMTQSPATLSLSPGERATLSCRSSKSLQNVNGNTYLYWFQQKPGQSPQLLIYR MSNLNSGVPDRFSGSGSGTEFTLTISSLEPEDFAVYYCMQHLEYPITFGAGTKLEI KRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC (SEQ ID NO: 12).

In some embodiments, the anti-CD19 antibody is tafasitamab.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered orally.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered intravenously. In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered subcutaneously.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered by intravenous infusion.

In some embodiments, the anti-CD19 antibody is administered intravenously.

In some embodiments, the anti-CD19 antibody is administered by intravenous infusion.

In some embodiments, the anti-CD19 antibody is administered by subcutaneous injection.

In some embodiments, the anti-CD19 antibody is administered subcutaneously.

In some embodiments, the cancer is a CD19 positive cancer.

In some embodiments, the cancer is a hematological malignancy.

In some embodiments, the cancer is a lymphoma or a leukemia.

In some embodiments, the cancer is a chronic lymphocytic leukemia or a non-Hodgkin's lymphoma.

In some embodiments, the cancer is a CD19 positive chronic lymphocytic leukemia or a CD19 positive non-Hodgkin's lymphoma.

In some embodiments, the cancer is a non-Hodgkin lymphoma. In some embodiments the human subject suffers from relapsed or refractory non-Hodkins lymphoma. In some embodiments the cancer is follicular lymphoma (FL), marginal zone lymphoma (MZL), mantle cell lymphoma (MCL), Diffuse large B-cell lymphoma (DLBCL), or Burkitt lymphoma.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every two weeks, once every week, twice a week, three times a week, or daily.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered twice a week.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every week.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered on days 1, 4, 8, and 11 of a 21 day cycle.

In some embodiments, the anti-CD19 antibody is administered once every two weeks, once every week, twice a week, three times a week, or daily.

In some embodiments, the anti-CD19 antibody is administered once every two weeks.

In some embodiments, the anti-CD19 antibody is administered once every week.

In some embodiments, the anti-CD19 antibody is administered once every four weeks.

In some embodiments, the anti-CD19 antibody is administered once every eight weeks.

In some embodiments, the anti-CD19 antibody is administered on Day 1 of a treatment cycle.

In some embodiments, the treatment cycle is 21 days or 28 days.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and the anti-CD19 antibody are administered simultaneously once every eight weeks, once every four weeks, once every two weeks, once every week, twice a week, three times a week, or daily.

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and the anti-CD19 antibody are administered simultaneously on days 1, 4, 8, and 11 of a 21 day cycle

In some embodiments, the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered once every two weeks, once every week, twice a week, three times a week, daily, or on days 1, 4, 8, and 11 of a 21 day cycle; and the anti-CD19 antibody is separately administered once every eight weeks, once every four weeks, once every two weeks, once every week, twice a week, three times a week, or daily.

In one aspect, the present disclosure relates to a kit comprising a medicament for use in treating cancer in a subject in need of such treatment. The kit comprises a medicament comprising an SAE inhibitor, and instructions for administering the SAE inhibitor and the one or more anti-CD19 antibodies or antibody fragments; or the kit comprises a medicament comprising the one or more anti-CD19 antibodies or antibody fragments, and instructions for administering the one or more anti-CD19 antibodies or antibody fragments and an SAE inhibitor. The kit can contain both a medicament comprising an SAE inhibitor and a medicament comprising one or more anti-CD20 antibodies, and instructions for administering the SAE inhibitor and the one or more anti-CD20 antibodies. The kit can also comprise one or more additional therapeutic agents.

In one aspect, the present disclosure relates to a medicament for use in treating cancer in a subject in need of such treatment. The medicament comprises an SAE inhibitor and one or more anti-CD19 antibodies or antibody fragments. The medicament can also comprise one or more additional therapeutic agents.

### BRIEF DESCRIPTION OF THE FIGURES

FIG. 1: Specific killing of lymphoma cell lines. PBMCs of 2-3 donors were tested in ADCC assay. Median values are shown.

FIG. 2: Degranulation of NK cells. Mean values with SD of four independent experiments are shown. Statistical analysis: paired t test. *p<0.05, **p<0.01, ***p<0.001, n.s. not significant.

FIG. 3: IFNγ release. Mean values with SD of four independent experiments with effector cells from 8 different donors are shown. Statistical analysis: paired t test. *p<0.05, ****p<0.0001.

FIG. 4: Phagocytosis of lymphoma cell lines. Mean values with SD of three independent experiments are shown. Statistical analysis: paired t test. *p<0.05, **p<0.01, ***p<0.001, n.s. not significant.

FIG. 5: Reduction of cell viability. Mean values with SD three independent experiments are shown. Statistical analysis: paired t test. *p<0.05, **p<0.01, ***p<0.001, n.s. not significant.

FIG. 6: Average tumor growth curves in a Daudi human xenograft tumor model.

FIG. 7: Average tumor growth curves in an OCI-Ly10 human xenograft tumor model.

FIG. 8: Average tumor growth curves in a WSU-DLCL2 human xenograft tumor model.

FIG. 9: Individual growth curves of Study 1 (Daudi).

### DETAILED DESCRIPTION

### Definitions

To facilitate an understanding of the present disclosure, a number of abbreviations, terms, and phrases are defined below.

Unless defined otherwise, all technical and scientific terms used herein have the same meaning as is commonly understood by one of skill in the art to which this disclosure belongs. All patents and publications referred to herein are incorporated by reference in their entirety.

As used herein, the term “cancer” refers to a cellular disorder characterized by uncontrolled or dysregulated cell proliferation, decreased cellular differentiation, inappropriate ability to invade surrounding tissue, and/or ability to establish new growth at ectopic sites. The term “cancer” includes solid tumors and non-solid tumors, such as, for example, hematological tumors. The term “cancer” encompasses diseases of skin, tissues, organs, bone, cartilage, blood, and vessels. The term “cancer” further encompasses primary and metastatic cancers.

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12. The term also encompasses naturally occurring variants of CD19, e.g., splice variants, allelic variants, and isoforms.

In an embodiment human CD19 has the amino acid sequence of:

(SEQ ID NO: 13)
MPPPRLLFFLLFLTPMEVRPEEPLVVKVEEGDNAVLQCLKGTSDGP
TQQLTWSRESPLKPFLKLSLGLPGLGIHMRPLAIWLFIFNVSQQM
GGFYLCQPGPPSEKAWQPGWTVNVEGSGELFRWNVSDLGGLGCGL
KNRSSEGPSSPSGKLMSPKLYVWAKDRPEIWEGEPPCLPPRDSLN
QSLSQDLTMAPGSTLWLSCGVPPDSVSRGPLSWTHVHPKGPKSLL
SLELKDDRPARDMWVMETGLLLPRATAQDAGKYYCHRGNLTMSFH
LEITARPVLWHWLLRTGGWKVSAVTLAYLIFCLCSLVGILHLQRA
LVLRRKRKRMTDPTRRFFKVTPPPGSGPQNQYGNVLSLPTPTSGL
GRAQRWAAGLGGTAPSYGNPSSDVQADGALGSRSPPGVGPEEEEG
EGYEEPDSEEDSEFYENDSNLGQDQLSQDGSGYENPEDEPLGPED
EDSFSNAESYENEDEELTQPVARTMDFLSPHGSAWDPSREATSLG
SQSYEDMRGILYAAPQLRSIRGQPGPNHEEDADSYENMDNPDGPD
PAWGGGGRMGTWSTR

“MOR208” and “XmAb 5574” and “tafasitamab” are used as synonyms for the anti-CD19 antibody according to Table 1. Table 1 provides the amino acid sequences of MOR208/tafasitamab. The MOR208 antibody is described in U.S. patent application Ser. No. 12/377,251, which is incorporated by reference in its entirety.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses polyclonal antibodies, monoclonal antibodies, antibody fragments (such as Fab, Fab′, F(ab′)2, and Fv fragments), single chain Fv (scFv) mutants, multispecific antibodies such as bispecific antibodies for example generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology. The different classes of immunoglobulins have different and well-known subunit structures and three-dimensional configurations. Antibodies can be naked or conjugated to other molecules such as toxins, radioisotopes, etc.

The term “anti-CD19 antibody” or “an antibody that binds to CD19” refers to an antibody that is capable of binding CD19 with sufficient affinity such that the antibody is useful as a diagnostic and/or therapeutic agent in targeting CD19.

A “monoclonal antibody” refers to a homogeneous or substantially homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “chimeric antibodies” refers to antibodies wherein the amino acid sequence of the immunoglobulin molecule is derived from two or more species.

Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

As used herein, the term “effective amount” or “therapeutically effective amount” refers to an amount of a compound, or combination of one or more compounds that, when administered (either sequentially or simultaneously) elicits the desired biological or medicinal response, e.g., either destroys, slows or arrests the growth of the target cancer cells or slows or arrests the progression of the cancer in a patient. The therapeutically effective amount may vary depending upon the intended application (e.g., in vitro or in vivo), or the patient and disease condition being treated, and can depend on factors such as, e.g., the weight and age of the patient, the severity of the disease condition, the manner of administration and the like, which may readily be determined by one skilled in the art. The term “effective amount” or “therapeutically effective amount” also applies to an amount, such as one or more doses, that will induce a particular response in target cells, e.g., reduction of platelet adhesion and/or cell migration. For example, in some embodiments, the “therapeutically effective amount” as used herein refers to the amount of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and the amount of an anti-CD19 antibody that, when administered separately or in combination, have a beneficial effect. In some embodiments, the combined effect is additive. In some embodiments, the combined effect is synergistic. Further, it will be recognized by one skilled in the art that in the case of combination therapy, the amount of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof and/or the amount of the anti-CD19 antibody may be used in a “sub-therapeutic amount”, i.e., less than the therapeutically effective amount of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, or the anti-CD19 antibody alone.

In any form or composition, the administered dose(s) or the therapeutically effective (total) amount may be expressed as amount(s) of therapeutic substance(s) per patient as either based on (i) BSA, e.g., as mg/m2, or (ii) amount, e.g., as mg.

The term “about” refers to approximately, in the region of, roughly, or around. When the term “about” is used in conjunction with a number or a numerical range, it means that the number or numerical range referred to is an approximation within experimental variability (or within statistical experimental error), and thus the number or numerical range may vary from, for example, between 1% and 15% of the stated number or numerical range. In general, the term “about” is used herein to modify a numerical value above and below the stated value by a variance of +10%.

As used herein, “patient” generally means a mammal (e.g., human) who has been diagnosed with, exhibits symptoms of, or is otherwise believed to be afflicted with a disease, disorder, or condition (such as cancer). The term “patient” is used interchangeably herein with the term “subject”.

As used herein, “body surface area” (BSA) is calculated using a standard nomogram, e.g.,

$$BSA ( {m}^{2} ) = \sqrt{\frac{Ht ( cm ) \times Wt ( kg )}{3600}} or BSA = \sqrt{\frac{Ht ( in ) \times Wt ( in )}{3131}}$$

The terms “combination”, “pharmaceutical combination”, “combination administration”, “administered in combination,” and “administering a combination” refer to the administration of one therapy in addition to another therapy. Each component can be administered simultaneously or sequentially in any order at different points in time. As such, “in combination with” refers to administering of more than one pharmaceutically active ingredients (including, but not limited to, [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD19 antibody as disclosed herein) to a patient. Combination administration may refer to simultaneous administration or may refer to sequential administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD19 antibody as disclosed herein.

The terms “simultaneous” and “simultaneously” refer to the administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD19 antibody as disclosed herein, to a patient at the same time, or at two different time points that are separated by no more than about 2 hours. The simultaneous administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD19 antibody may be in a single dosage form or in separate dosage forms.

The terms “sequential” and “sequentially” refer to the administration of the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, and an anti-CD19 antibody as disclosed herein, to a patient at two different time points that are separated by more than about 2 hours, e.g., about 3 hours, about 4 hours, about 5 hours, about 8 hours, about 12 hours, 1 day, 2 days, 3 days, 4 days, 5 days, 6 days, 7 days or even longer.

The term “intermission” refers to a period that is subsequent to the administration of one or more particular pharmaceutically active ingredients to a patient in an intermittent regimen. Intermission refers to a rest period wherein a particular pharmaceutically active ingredient is not administered for at least one day.

The terms “synergy”, “synergism”, “synergistic” and “synergistic effect” which are used herein interchangeably refer to a situation where the combination of two or more agents produces a greater effect than the sum of the effects of each of the individual agents. The term encompasses not only a reduction in symptoms of the disorder to be treated, but also an improved side effect profile, improved tolerability, improved patient compliance, improved efficacy, or any other improved clinical outcome.

As used herein, the illustrative terms “include”, “such as”, “for example”, “e.g.” and the like (and variations thereof, e.g., “includes” and “including”, “examples”), unless otherwise specified, are intended to be non-limiting. That is, unless explicitly stated otherwise, such terms are intended to imply “but not limited to”, e.g., “including” means “including but not limited to”.

Unless otherwise stated, structures depicted herein are meant to include chemical entities which differ only in the presence of one or more isotopically enriched atoms. For example, chemical entities having the present structure except for the replacement of a hydrogen atom by a deuterium or tritium, or the replacement of a carbon atom by a 13C- or 14C-enriched carbon are within the scope of the invention.

Unless stereochemical configuration is denoted, structures depicted herein are meant to include all stereochemical forms of the structure, i.e., the R and S configurations for each asymmetric center. Therefore, unless otherwise indicated, single stereochemical isomers as well as enantiomeric, racemic and diastereomeric mixtures of the present chemical entities are within the scope of the invention. When a stereochemical configuration is denoted for a compound, the diastereoisomeric or enantiomeric excess of the compound is in some embodiments, at least 99.0%, 99.5%, 99.6%, 99.7%, 99.8% or 99.9%.

The present disclosure provides a combination treatment for patients with cancer. The combination treatment comprises administering to a subject in need thereof a therapeutically effective amount of at least one SAE inhibitor.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate, or a pharmaceutically acceptable salt thereof, having the following structure:

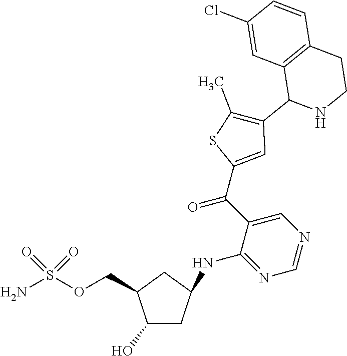 [SMILES: Cc1sc(C(=O)c2cncnc2N[C@@H]2C[C@H](COS(N)(=O)=O)[C@@H](O)C2)cc1C1NCCc2ccc(Cl)cc21]

Compound I-263a; [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate is also referred to herein as Compound I-263a.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate, or a pharmaceutically acceptable salt thereof.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate, or Compound I-263a.

SAE inhibitors, as disclosed herein, are described, for example, in US 2016/0009744 and U.S. Pat. No. 9,695,154 which is hereby incorporated by reference in its entirety. They may be prepared by methods known to one skilled in the art and/or according to the methods described in US 2016/0009744 and U.S. Pat. No. 9,695,154 . . . . Central to the mechanism of action of useful SAE inhibitors, such as Compound I-263a, in combinations and methods of the present disclosure is production of type 1 IFNs and induction of an innate immune response with activation of both natural killer (NK) cells and macrophages. Biochemical assays have demonstrated that Compound I-263a is a mechanism-based inhibitor of SUMO-activating enzyme that potently inhibits enzyme activity by forming a covalent adduct with SUMO. Strong selectivity for SUMO-activating enzyme was observed over the other closely related ubiquitin-activating enzymes ubiquitin-activating enzyme, Nedd8-activating enzyme, and autophagy related 7 enzyme. Selective and potent inhibition of SUMO-activating enzyme and SUMOylation by Compound I-263a has been demonstrated in cultured mouse and human tumor cell lines and the antiproliferative activity of Compound I-263a has been determined in a panel of 7 mouse hematologic and solid tumor cell lines.

In some embodiments, the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a crystalline form thereof.

In some embodiments, the SAE inhibitor or a pharmaceutical salt thereof is crystalline form 1 of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a), as described in U.S. published application number US 2016/0009744.

In some embodiments, the SAE inhibitor or a pharmaceutical salt thereof is crystalline form 2 of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a), as described in U.S. published application number US 2016/0009744.

In some embodiments, the SAE inhibitor or a pharmaceutical salt thereof is crystalline form 3 of [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a), described in U.S. published application number US 2016/0009744.

The present disclosure provides a combination treatment that includes, inter alia, administering to a subject in need thereof a therapeutically effective amount of at least one anti-CD19 antibody (e.g., tafasitamab).

The use of anti-CD19 antibodies in non-specific B cell lymphomas is discussed in WO2007076950 (US2007154473), which are both incorporated by reference in their entirety. The use of a CD19-antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety.

Additional antibodies specific for CD19 are described in WO2005012493 (U.S. Pat. No. 7,109,304), WO2010053716 (U.S. Ser. No. 12/266,999) (Immunomedics); WO2007002223 (US U.S. Pat. No. 8,097,703) (Medarex); WO2008022152 (Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (Ser. No. 12/710,442) (Glenmark Pharmaceuticals), WO2012010562 and WO2012010561 (International Drug Development), WO2011147834 (Roche Glycart), and WO2012156455 (Sanofi), which are all incorporated by reference in their entireties.

The dose of an antibody comprised in a pharmaceutical composition according to the present disclosure administered to a patient may vary depending upon the age and the size of the patient, symptoms, conditions, route of administration, and the like. The dose is typically calculated according to body weight, or body surface area, age, or per individual. Depending on the severity of the condition, the frequency and the duration of the treatment can be adjusted. Effective dosages and schedules for administering pharmaceutical compositions comprising antibodies or antibody fragments specific for CD19 may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

The pharmaceutical composition may include dosage forms for intravenous, subcutaneous, intracutaneous and intramuscular injections. These injectable preparations may be prepared by known methods. For example, the injectable preparations may be prepared, e.g., by dissolving, suspending or emulsifying the antibody described above or its salt in a sterile aqueous medium or an oily medium conventionally used for injections. Exemplary pharmaceutical compositions comprising an antibody specific for CD19 that can be used in the context of the present disclosure are disclosed, e.g., in WO2008/022152 or WO2018/002031 which are both incorporated by reference in their entireties.

In certain ways of administration used with embodiments of the present disclosure, e.g. intravenous administration, it is preferred to administer drug depending on the body weight of the patient. In other ways of administration used with embodiments of the present disclosure, e.g. subcutaneous administration, it is preferred to administer drug at a flat, fixed does. The skilled person is aware of which dose in one way of administration is equivalent or substantially equivalent to another dose in another way of administration. For example, the pharmacodynamics of a specific drug are typically taken into account in a reasoned decision to administer a drug in the required from and at a required, efficacious dose. The antibody which is administered according to the present disclosure can be administered to the patient in a therapeutically effective amount.

In some embodiments, the present disclosure relates to a method of treating a cancer in a patient by administering to a patient in need of said treatment a combination of an SAE inhibitor or pharmaceutically acceptable salt thereof and one or more anti-CD19 antibodies.

In some embodiments, the present disclosure relates to a method of treating a cancer by administering to a patient in need of said treatment a combination of an SAE inhibitor and an anti-CD19 antibody.

In some embodiments, the present disclosure relates to the use of an SAE inhibitor in combination with an anti-CD19 antibody for the treatment of a cancer in a patient.

In some embodiments, the present disclosure relates to a composition comprising an SAE inhibitor for use in treating a cancer in a patient, wherein the patient is also treated with an anti-CD19 antibody. In some aspects, the disclosure relates to a composition comprising an SAE inhibitor for use in treating a disorder which is cancer in a patient, wherein the SAE inhibitor is in combination with the anti-CD19 antibody. In some embodiments, the SAE inhibitor can be administered simultaneously or sequentially with the anti-CD19 antibody.

In some embodiments, the present disclosure relates to methods of treating a cancer comprising administering to a patient in need of such treatment, a therapeutically effective amount of a combination of an SAE inhibitor and an anti-CD19 antibody.

In some embodiments, the present disclosure relates to a method of treating a cancer by administering to a patient a combination of Compound I-263a, or pharmaceutically acceptable salt thereof, and an anti-CD19 antibody.

In another aspect, the present disclosure relates to the use of Compound I-263a, or a pharmaceutically acceptable salt thereof, in combination with an anti-CD19 antibody for the treatment of a cancer.

In some embodiments, the methods of treating a cancer, as described herein, can include a combination of an SAE inhibitor, an anti-CD19 antibody, and one or more additional therapeutic agents. In some embodiments, the one or more additional therapeutic agents can be chemotherapeutic agents. In some embodiments, the one or more additional therapeutic agents can include, but are not limited to lenalidomide, fludarabine, cyclophosphamide, doxorubicin, vincristine, methotrexate anthracycline-based chemotherapeutic agents, prednisone, methylprednisolone, glucocorticoids, Ibritumomab tiuxetan, acetaminophen, antihistamines, and combinations thereof.

In some embodiments, the cancer is a CD19-positive cancer.

In some embodiments, the cancer is a hematological cancer. Non-limiting examples of hematologic cancers include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's lymphoma (HL); non-Hodgkin's lymphoma (NHL), including B-cell lymphoma, T-cell lymphoma, follicular lymphoma (FL), marginal zone lymphoma (MZL), mantle cell lymphoma (MCL), diffuse large B-cell lymphoma (DLBCL), and Burkitt lymphoma; multiple myeloma (MM); amyloidosis; Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes. In some embodiments, the cancer is chronic lymphocytic leukemia (CLL), Hodgkin's lymphoma, or non-Hodgkin's lymphoma including follicular lymphoma (FL), marginal zone lymphoma (MZL), mantle cell lymphoma (MCL), diffuse large B-cell lymphoma (DLBCL) and Burkitt lymphoma.

In some embodiments, the cancer is chronic lymphocytic leukemia. In some embodiments, the cancer is CD19-positive chronic lymphocytic leukemia.

In some embodiments, the cancer is a non-Hodgkin's lymphoma, including follicular lymphoma (FL), marginal zone lymphoma (MZL), mantle cell lymphoma (MCL), Diffuse large B-cell lymphoma (DLBCL), and Burkitt lymphoma. In some embodiments, the cancer is a B-cell lymphoma. In some embodiments, the cancer is a CD19-positive non-Hodgkin's lymphoma. In some embodiments, the cancer is a CD19-positive aggressive non-Hodgkin lymphoma. In some embodiments, the cancer is a CD19-positive indolent non-Hodgkin lymphoma. In some embodiments, the cancer is relapsed or refractory non-Hodgkin lymphoma. In some embodiments, the cancer is relapsed or refractory CD19-positive aggressive non-Hodgkin lymphoma. In some embodiments, the cancer is relapsed or refractory CD19-positive indolent non-Hodgkin lymphoma.

In some embodiments, the cancer is relapsed. In some embodiments, relapsed cancer is cancer which has returned after a period of time in which no cancer could be detected.

In some embodiments, the cancer is refractory. In some embodiments, refractory cancer does not respond to cancer treatment; it is also known as resistant cancer. In some embodiments, the cancer is resistant to rituximab. In some embodiments, the cancer does not respond to the treatment of rituximab. In some embodiments, the cancer is rituximab-resistant recurrent cancer. In some embodiments, the patient has become refractory to a rituximab-containing regimen. In some embodiments, the tumor is unresectable. In some embodiments, an unresectable tumor is unable to be removed by surgery. In some embodiments, the cancer has not been previously treated. In some embodiments, the cancer is locally advanced. In some embodiments, “locally advanced” refers to cancer that is somewhat extensive but still confined to one area. In some instances, “locally advanced” may refer to a small tumor that hasn't spread but has invaded nearby organs or tissues that make it difficult to remove with surgery alone. In some embodiments, the cancer is metastatic. In some embodiments, metastatic cancer is a cancer that has spread from the part of the body where it started (the primary site) to one or more other parts of the body.

In some embodiments, the patient has relapsed or refractory CD19-positive non-Hodgkin lymphoma. In some embodiments, the patient has both CD19-positive non-Hodgkin lymphoma and relapsed or refractory non-Hodgkin lymphoma.

In some embodiments, the patient has relapsed or refractory CD19-positive aggressive non-Hodgkin lymphoma. In some embodiments, the patient has relapsed or refractory CD19-positive aggressive non-Hodgkin lymphoma and has progressed on at least one prior treatment regimen.

In some embodiments, the patient has relapsed or refractory CD19-positive indolent non-Hodgkin lymphoma. In some embodiments, the patient has relapsed or refractory CD19-positive indolent non-Hodgkin lymphoma and has progressed on at least two prior treatment regimens. In some embodiments, the patient has relapsed or refractory CD19-positive indolent non-Hodgkin lymphoma and is refractory to any anti-CD20 monoclonal antibody or any anti-CD19 monoclonal antibody. In some embodiments, the patient has relapsed or refractory CD19-positive indolent non-Hodgkin lymphoma and has progressed on at least two prior treatment regimens and is refractory to any anti-CD20 monoclonal antibody.

In some embodiments, the present disclosure relates to a medicament for use in treating a cancer in a patient in need of such treatment. The medicament comprises an SAE inhibitor and an anti-CD19 antibody, and is in single dosage form or in separate dosage forms.

In some embodiments, the medicaments, as described herein, can include a combination of an SAE inhibitor, an anti-CD19 antibody, and optionally one or more additional therapeutic agents.

In some embodiments, the present disclosure relates to the use of an SAE inhibitor in the manufacture of a medicament for treating a cancer, wherein the SAE inhibitor is administered with an anti-CD19 antibody, and wherein the medicament is in single dosage form or in separate dosage forms. In some embodiments, the SAE inhibitor is administered with an anti-CD19 antibody and one or more additional therapeutic agents.

In some embodiments, the present disclosure relates to the use of an SAE inhibitor for the manufacture of a medicament in treating a cancer in a patient, wherein the patient is also treated with an anti-CD19 antibody, and optionally one or more additional therapeutic agents. In some embodiments, the SAE inhibitor may be administered simultaneously or sequentially with the anti-CD19 antibody. In some aspects, the present disclosure relates to the use of an SAE inhibitor for the manufacture of a medicament in treating a cancer in a patient, wherein the SAE inhibitor is in combination with an anti-CD19 antibody, and optionally one or more additional therapeutic agents. In some embodiments, the SAE inhibitor is in the same composition as the anti-CD19 antibody. In some embodiments, the SAE inhibitor is in a separate composition as the anti-CD19 antibody. In some embodiments, the SAE inhibitor is in the same composition as one or more additional therapeutic agents. In some embodiments, the SAE inhibitor is in the same composition as the anti-CD19 antibody, and optionally one or more additional therapeutic agents. In some embodiments, the SAE inhibitor is in a separate composition as one or more additional therapeutic agents. In some embodiments, the SAE inhibitor is in a separate composition as the anti-CD19 antibody, and optionally one or more additional therapeutic agents.

In another aspect, the present disclosure relates to the use of Compound I-263a, or a pharmaceutically acceptable salt thereof in combination with an anti-CD19 antibody in the manufacture of a medicament for use in treating a cancer. In some embodiments, the present disclosure relates to the use of Compound I-263a, or a pharmaceutically acceptable salt thereof in combination with an anti-CD19 antibody, and optionally one or more additional therapeutic agents in the manufacture of a medicament for use in treating a cancer.

In another aspect, the present disclosure relates to the use of Compound I-263a, or a pharmaceutically acceptable salt thereof, in the manufacture of a medicament for treating a cancer, wherein Compound I-263a or a pharmaceutically acceptable salt thereof is administered with an anti-CD19 antibody, and optionally one or more additional therapeutic agents.

In some embodiments, the one or more additional therapeutic agents can be chemotherapeutic agents. In some embodiments, the one or more additional therapeutic agents can include, but are not limited to, fludarabine, cyclophosphamide, doxorubicin, vincristine, methotrexate anthracycline-based chemotherapeutic agents, prednisone, methylprednisolone, glucocorticoids, Ibritumomab tiuxetan, acetaminophen, antihistamines, and combinations thereof.

In one aspect, the present disclosure relates to an anti-CD19 antibody for use in the treatment of a cancer, wherein said anti-CD19 antibody is administered in combination with an SAE inhibitor to a subject in need of such treatment.

In one aspect, the present disclosure relates to an anti-CD19 antibody for use in the treatment of a cancer, wherein said anti-CD19 antibody is administered in combination with [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof. Compound I-263a is also sometimes referred to herein as TAK-981.

In some embodiments, the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In some embodiments, the anti-CD19 antibody comprises a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3) and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

In some embodiments, the anti-CD19 antibody comprises a heavy chain variable region of EVOLVESGGGLVKPGGSLKLSCAASGYTFTSYVMHWVRQAPGKGLEWIGYINPY NDGTKYNEKFQGRVTISSDKSISTAYMELSSLRSEDTAMYYCARGTYYYGTRVF DYWGQGTLVTVSS (SEQ ID NO: 7) and a light chain variable region of DIVMTQSPATLSLSPGERATLSCRSSKSLQNVNGNTYLYWFQQKPGQSPQLLIYR MSNLNSGVPDRFSGSGSGTEFTLTISSLEPEDFAVYYCMQHLEYPITFGAGTKLEI K (SEQ ID NO: 8).

In some embodiments, the anti-CD19 antibody has effector function. In another aspect the antibody or antibody fragment specific for CD19 has an enhanced effector function. In one embodiment the effector function is ADCC. In one embodiment the antibody or antibody fragment specific for CD19 has an enhanced ADCC activity. In a further embodiment the antibody or antibody fragment specific for CD19 comprises an Fc domain comprising an amino acid substitution at position S239 and/or 1332, wherein the numbering is according to the EU index as in Kabat.

In some embodiments, the anti-CD19 antibody comprises a heavy chain constant region of ASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCP PCPAPELLGGPDVFLFPPKPKDTLMISRTPEVTCVVVDVSHEDPEVQFNWYVDGV EVHNAKTKPREEQFNSTFRVVSVLTVVHQDWLNGKEYKCKVSNKALPAPEEKTI SKTKGQPREPQVYTLPPSREEMTKNQVSLTCL VKGFYPSDIA VEWESNGQPENNY KTTPPMLDSDGSFFLYSKLTVDKSRWQQGNVFSCSVMHEALHNHYTQKSLSLSP GK (SEQ ID NO: 9).

In some embodiments, the anti-CD19 antibody comprises a light chain constant region of RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 10).

In some embodiments, the anti-CD19 antibody comprises a heavy chain constant region of ASTKGPSVFPLAPSSKSTSGGTAALGCLVKDYFPEPVTVSWNSGALTSGVHTFPA VLQSSGLYSLSSVVTVPSSSLGTQTYICNVNHKPSNTKVDKKVEPKSCDKTHTCP PCPAPELLGGPDVFLFPPKPKDTLMISRTPEVTCVVVDVSHEDPEVQFNWYVDGV EVHNAKTKPREEQFNSTFRVVSVLTVVHQDWLNGKEYKCKVSNKALPAPEEKTI SKTKGQPREPQVYTLPPSREEMTKNQVSLTCLVKGFYPSDIA VEWESNGQPENNY KTTPPMLDSDGSFFLYSKLTVDKSRWQQGNVFSCSVMHEALHNHYTQKSLSLSP GK (SEQ ID NO: 9) and a light chain constant region of RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 10).

In some embodiments, the anti-CD19 antibody comprises a heavy chain region of EVOLVESGGGLVKPGGSLKLSCAASGYTFTSYVMHWVRQAPGKGLEWIGYINPY NDGTKYNEKFQGR VTISSDKSISTAYMELSSLRSEDTAMYYCARGTYYYGTR VF DYWGQGTLVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK KVEPKSCDKTHTCPPCPAPELLGGPDVFLFPPKPKDTLMISRTPEVTCVVVDVSHE DPEVQFNWYVDGVEVHNAKTKPREEQFNSTFRVVSVLTVVHQDWLNGKEYKC KVSNKALPAPEEKTISKTKGQPREPQVYTLPPSREEMTKNQVSLTCL VKGFYPSDI AVEWESNGQPENNYKTTPPMLDSDGSFFLYSKLTVDKSRWQQGNVFSCSVMHE ALHNHYTQKSLSLSPGK (SEQ ID NO: 11) and a light chain region of DIVMTQSPATLSLSPGERATLSCRSSKSLQNVNGNTYLYWFQQKPGQSPQLLIYR MSNLNSGVPDRFSGSGSGTEFTLTISSLEPEDFAVYYCMQHLEYPITFGAGTKLEI KRTVAAPSVFIFPPSDEQLKSGTASVVCLLNNFYPREAKVQWKVDNALQSGNSQ ESVTEQDSKDSTYSLSSTLTLSKADYEKHK VYACEVTHQGLSSPVTKSFNRGEC (SEQ ID NO: 12).

In some embodiments, the anti-CD19 antibody is tafasitamab.

Compound I-263a or a pharmaceutically acceptable salt thereof, may be administered in combination with the anti-CD19, and optionally one or more additional therapeutic agents, in a single dosage form or as a separate dosage forms. In some embodiments, when administered as a separate dosage form, the anti-CD19 antibody may be administered prior to, at the same time as, or following administration of I-263a or a pharmaceutically acceptable salt thereof. In some embodiments, when administered as a separate dosage form, one or more doses of I-263a or a pharmaceutically acceptable salt thereof, may be administered prior to the anti-CD19. In some embodiments, the anti-CD20 antibody is administered prior to the administration of Compound I-263a or a pharmaceutically acceptable salt thereof. As used herein, the administration in “combination” of Compound I-263a or a pharmaceutically acceptable salt thereof, an anti-CD19 antibody, and optionally one or more additional therapeutic agents refers not only to simultaneous or sequential administration of the agents, but also to the administration of the agents during a single treatment cycle, as understood by one skilled in the art. When Compound I-263a or a pharmaceutically acceptable salt thereof is administered in combination with the anti-CD19 antibody, and optionally one or more additional therapeutic agents, a therapeutically effective amount of the combination is administered.

The SAE inhibitor may be administered by any method known to one skilled in the art. For example, in some embodiments, the SAE inhibitor may be administered in the form of a pharmaceutical composition of the SAE inhibitor and a pharmaceutically acceptable carrier, such as those described herein. In some embodiments, the pharmaceutical composition is suitable for oral administration. In some embodiments, the pharmaceutical composition is a tablet or a capsule that is suitable for oral administration. In some other embodiments, the pharmaceutical composition is a liquid dosage form suitable for oral administration. In some embodiments, the pharmaceutical composition is suitable for intravenous administration. In some embodiments, the pharmaceutical composition is suitable for subcutaneous administration. In some embodiments, these compositions optionally further comprise one or more additional therapeutic agents.

The anti-CD19 antibody may be administered by any method known to one skilled in the art. In some embodiments, the anti-CD19 antibody is administered intravenously (IV). In some embodiments, the anti-CD19 antibody is administered subcutaneously (SC). In some embodiments, the anti-CD19 antibody is administered orally. For example, the anti-CD19 antibody may be administered in the form of a second composition, in some embodiments, a pharmaceutical composition of the anti-CD19 antibody and a pharmaceutically acceptable carrier, such as those described herein. In some aspects, the pharmaceutical composition is suitable for oral administration. In some embodiments, the pharmaceutical composition is a tablet or a capsule that is suitable for oral administration. In some other embodiments, the pharmaceutical composition is a liquid dosage form suitable for oral administration. In some embodiments, these compositions optionally further comprise one or more additional therapeutic agents.

The amounts or suitable doses of the methods of this disclosure depends upon a number of factors, including the nature of the severity of the condition to be treated, the particular inhibitor, the route of administration and the age, weight, general health, and response of the individual patient. In some embodiments, the suitable dose level is one that achieves a therapeutic response as measured by tumor regression, or other standard measures of disease progression, progression free survival or overall survival. In some embodiments, the suitable dose level is one that achieves this therapeutic response and also minimizes any side effects associated with the administration of the therapeutic agent. The suitable dose levels may be ones that prolong the therapeutic response and/or prolong life.

It will be understood that a suitable dose of the SAE inhibitor, the anti-CD19 antibody, and optionally one or more additional therapeutic agents may be taken at any time of the day or night. In some embodiments, a suitable dose of each agent is taken in the morning. In some other embodiments, a suitable dose of each agent is taken in the evening. In some embodiments, a suitable dose of each of the agents is taken both in the morning and the evening. It will be understood that a suitable dose of each agent may be taken with or without food. In some embodiments a suitable dose of an agent is taken with a meal. In some embodiments a suitable dose of an agent is taken while fasting.

In some embodiments, Compound I-263a or a pharmaceutically acceptable salt thereof is administered on a daily schedule. In some embodiments, Compound I-263a or a pharmaceutically acceptable salt thereof is administered every other day. In some embodiments, Compound I-263a or a pharmaceutically acceptable salt thereof is administered once every three days. In some embodiments, Compound I-263a or a pharmaceutically acceptable salt thereof is administered on a twice-weekly schedule. In some embodiments, Compound I-263a or a pharmaceutically acceptable salt thereof is administered on a three times a week schedule. In some embodiments, Compound I-263a or a pharmaceutically acceptable salt thereof is administered on a weekly schedule. In some embodiments, Compound I-263a or a pharmaceutically acceptable salt thereof is administered on a once every two weeks schedule.

In some embodiments, Compound I-263a or a pharmaceutically acceptable salt thereof is administered at least 3 times on alternate days within a 7-day cycle. In some embodiments, Compound I-263a or a pharmaceutically acceptable salt thereof is administered on day 1 and day 4 of a 7-day cycle. In some embodiments, Compound I-263a or a pharmaceutically acceptable salt thereof is administered on consecutive days in a 7-day cycle followed by an intermission. In some embodiments, Compound I-263a or a pharmaceutically acceptable salt thereof is administered for 2 consecutive days followed by an intermission of 5 consecutive days for at least one 7-day cycle. In some embodiments, Compound I-263a or a pharmaceutically acceptable salt thereof is administered for 3 consecutive days followed by an intermission of 4 consecutive days for at least one 7-day cycle. In some embodiments, Compound I-263a or a pharmaceutically acceptable salt thereof is administered for 4 consecutive days followed by an intermission of 3 consecutive days for at least one 7-day cycle. In some embodiments, Compound I-263a or a pharmaceutically acceptable salt thereof is administered for 5 consecutive days followed by an intermission of 2 consecutive days for at least one 7-day cycle. In some embodiments, there will be periods of rest between one or more of the 7-day treatment cycles. In some embodiments, there will be a 7-day rest between one or more of the 7-day treatment cycles.

The present description contemplates administration of the drug for one or more treatment cycles, for example, 1, 2, 3, 4, 5, 6, or more, treatment cycles. In some embodiments, a treatment cycle is about 7 days to about 56 days, or more. In some embodiments, a treatment cycle is 7 days, 14 days, 21 days, 28 days, 35 days, 42 days, 49 days, or 56 days. In some embodiments, a treatment cycle is 21 days or 28 days. In some embodiments, there will be periods of rest within or between one or more of the treatment cycles. For example, in some embodiments, there will be a period of rest at the end of the treatment cycle. In some embodiments, there will be a period of rest between the second and third treatment cycle but not the first and second treatment cycle. In another embodiment, there might be a period of rest between the first and second treatment cycle but not the second and third treatment cycle. Dosing schedules include, for example, administering the SAE inhibitor once during a treatment schedule, e.g., on day 1 of a 21 day cycle, twice during a treatment cycle, e.g., on days 1 and 15 of a 21 day cycle or on days 1 and 15 of a 28 day cycle, three times during a treatment cycle, e.g., on days 1, 8 and 15 of a 21 day cycle or on days 1, 8 and 15 of a 28 day cycle, and four times during a treatment cycle, e.g., on days 1, 4, 8, and 11 of a 21 day cycle or on days 1, 4, 8, and 11 of a 28 day cycle. Other dosage schedules are encompassed by the present invention.

In some embodiments, Compound I-263a or a pharmaceutically acceptable salt thereof is administered within a 21-day cycle. In some embodiments, Compound I-263a or a pharmaceutically acceptable salt thereof is administered at least two times within a 21-day cycle. In some embodiments, Compound I-263a or a pharmaceutically acceptable salt thereof is administered on day 1 within a 21-day cycle. In some embodiments, Compound I-263a or a pharmaceutically acceptable salt thereof is administered on day 8 within a 21-day cycle. In some embodiments, Compound I-263a or a pharmaceutically acceptable salt thereof is administered on days 1 and 8 within a 21-day cycle.

In some embodiments, Compound I-263a or a pharmaceutically acceptable salt thereof is administered for a duration of 1 year or less. In some embodiments, Compound I-263a or a pharmaceutically acceptable salt thereof is administered for a duration of 1 year or more.

In some embodiments, the amount of Compound I-263a or a pharmaceutically acceptable salt thereof that is administered on each day of dosing is about 0.5 mg to about 200 mg. In some embodiments, the amount of Compound I-263a or a pharmaceutically acceptable salt thereof that is administered on each day of dosing is about 0.5 mg to about 100 mg. In some embodiments, the amount of Compound I-263a or a pharmaceutically acceptable salt thereof that is administered on each day of dosing is about 0.5 mg to about 50 mg. In some embodiments, the amount of Compound I-263a or a pharmaceutically acceptable salt thereof that is administered on each day of dosing is about 0.5 mg to about 10 mg. In some embodiments, the amount of Compound I-263a or a pharmaceutically acceptable salt thereof that is administered on each day of dosing is about 0.5 mg to about mg. In some embodiments, the amount of Compound I-263a or a pharmaceutically acceptable salt thereof that is administered on each day of dosing is about 1 mg to about 3 mg. In some embodiments, the amount of Compound I-263a or a pharmaceutically acceptable salt thereof that is administered on each day of dosing is about 2 mg to about 5 mg. In some embodiments, the amount of Compound I-263a or a pharmaceutically acceptable salt thereof that is administered on each day of dosing is about 5 mg to about mg. In some embodiments, the amount of Compound I-263a or a pharmaceutically acceptable salt thereof that is administered on each day of dosing is about 5 mg to about mg. In some embodiments, the amount of Compound I-263a or a pharmaceutically acceptable salt thereof that is administered on each day of dosing is about 10 mg to about mg. In some embodiments, the amount of Compound I-263a or a pharmaceutically acceptable salt thereof that is administered on each day of dosing is about 15 mg to about mg. In some embodiments, the amount of Compound I-263a or a pharmaceutically acceptable salt thereof that is administered on each day of dosing is between about 20 mg to about 30 mg. In some embodiments, the amount of Compound I-263a or a pharmaceutically acceptable salt thereof that is administered on each day of dosing is between about 25 mg to about 35 mg. In some embodiments, the amount of Compound I-263a or a pharmaceutically acceptable salt thereof that is administered on each day of dosing is between about 30 mg to about 40 mg. In some embodiments, the amount of Compound I-263a or a pharmaceutically acceptable salt thereof that is administered on each day of dosing is between about 35 mg to about 45 mg. In some embodiments, the amount of Compound I-263a or a pharmaceutically acceptable salt thereof that is administered on each day of dosing is between about 40 mg to about 50 mg. In some embodiments, the amount of Compound I-263a or a pharmaceutically acceptable salt thereof that is administered on each day of dosing is between about 55 mg to about 65 mg. In some embodiments, the amount of Compound I-263a or a pharmaceutically acceptable salt thereof that is administered on each day of dosing is between about 50 mg to about 100 mg. In some embodiments, the amount of Compound I-263a or a pharmaceutically acceptable salt thereof that is administered on each day of dosing is between about 90 mg to about 150 mg. In some embodiments, the amount of Compound I-263a or a pharmaceutically acceptable salt thereof that is administered on each day of dosing is between about 140 mg to about 200 mg. In some embodiments, the amount of Compound I-263a or a pharmaceutically acceptable salt thereof that is administered on each day of dosing is about 0.5 mg. In some embodiments, the amount of Compound I-263a or a pharmaceutically acceptable salt thereof that is administered on each day of dosing is about 1 mg. In some embodiments, the amount of Compound I-263a or a pharmaceutically acceptable salt thereof that is administered on each day of dosing is about 2 mg. In some embodiments, the amount of Compound I-263a or a pharmaceutically acceptable salt thereof that is administered on each day of dosing is about 3 mg. In some embodiments, the amount of Compound I-263a or a pharmaceutically acceptable salt thereof that is administered on each day of dosing is about 4 mg. In some embodiments, the amount of Compound I-263a or a pharmaceutically acceptable salt thereof that is administered on each day of dosing is about 6 mg. In some embodiments, the amount of Compound I-263a or a pharmaceutically acceptable salt thereof that is administered on each day of dosing is about 8 mg. In some embodiments, the amount of Compound I-263a or a pharmaceutically acceptable salt thereof that is administered on each day of dosing is about 10 mg. In some embodiments, the amount of Compound I-263a or a pharmaceutically acceptable salt thereof that is administered on each day of dosing is about 12 mg. All dosing amounts refer to the amount of Compound I-263a administered, and do not include the weight amount of any pharmaceutically acceptable salt.

In some embodiments, the amount of Compound I-263a or a pharmaceutically acceptable salt thereof that is administered on each day of dosing is about 1 mg. In some embodiments, the amount of Compound I-263a or a pharmaceutically acceptable salt thereof that is administered on each day of dosing is about 3 mg. In some embodiments, the amount of Compound I-263a or a pharmaceutically acceptable salt thereof that is administered on each day of dosing is about 6 mg. In some embodiments, the amount of Compound I-263a or a pharmaceutically acceptable salt thereof that is administered on each day of dosing is about 10 mg. In some embodiments, the amount of Compound I-263a or a pharmaceutically acceptable salt thereof that is administered on each day of dosing is about 15 mg. In some embodiments, the amount of Compound I-263a or a pharmaceutically acceptable salt thereof that is administered on each day of dosing is about 25 mg. In some embodiments, the amount of Compound I-263a or a pharmaceutically acceptable salt thereof that is administered on each day of dosing is about 40 mg. In some embodiments, the amount of Compound I-263a or a pharmaceutically acceptable salt thereof that is administered on each day of dosing is about 60 mg. In some embodiments, the amount of Compound I-263a or a pharmaceutically acceptable salt thereof that is administered on each day of dosing is about 90 mg. In some embodiments, the amount of Compound I-263a or a pharmaceutically acceptable salt thereof that is administered on each day of dosing is about 120 mg. In some embodiments, the amount of Compound I-263a or a pharmaceutically acceptable salt thereof that is administered on each day of dosing is about 160 mg.

In some embodiments, the anti-CD19 antibody is administered on a daily schedule. In some embodiments, the anti-CD19 antibody is administered every other day. In some embodiments, the anti-CD19 antibody is administered once every three days. In some embodiments, the anti-CD19 antibody is administered on a twice-weekly schedule. In some embodiments, anti-CD19 antibody is administered on a three times a week schedule. In some embodiments, the anti-CD19 antibody is administered on a weekly schedule. In some embodiments, the anti-CD19 antibody is administered on a once every two weeks schedule. In some embodiments, the anti-CD19 antibody is administered on a once every three weeks schedule. In some embodiments, the anti-CD19 antibody is administered on a once every four weeks schedule.

In some embodiments, the anti-CD19 antibody is administered at least 3 times on alternate days within a 7-day cycle. In some embodiments, the anti-CD19 antibody is administered on day 1 and day 4 of a 7-day cycle. In some embodiments, the anti-CD19 antibody is administered on consecutive days in a 7-day cycle followed by an intermission. In some embodiments, the anti-CD19 antibody is administered for 2 consecutive days followed by an intermission of 5 consecutive days for at least one 7-day cycle. In some embodiments, the anti-CD19 antibody is administered for 3 consecutive days followed by an intermission of 4 consecutive days for at least one 7-day cycle. In some embodiments, the anti-CD19 antibody is administered for 4 consecutive days followed by an intermission of 3 consecutive days for at least one 7-day cycle. In some embodiments, the anti-CD19 antibody is administered for 5 consecutive days followed by an intermission of 2 consecutive days for at least one 7-day cycle.

The SAE inhibitors and the anti-CD19 antibodies used in the methods and kits described herein can be formulated into pharmaceutical compositions suitable for administration. The pharmaceutical compositions may comprise pharmaceutically acceptable excipients. A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21^{st} Ed., A. R. Gennaro (Lippincott, Williams & Wilkins, Baltimore, MD), 2006; incorporated by reference in its entirety)

Any of the therapeutic agents described herein may be in the form of a pharmaceutically acceptable salt. In some embodiments, such salts are derived from inorganic or organic acids or bases. For reviews of suitable salts, see, e.g., Berge et al., J. Pharm. Sci., 1977, 66, 1-19 and Remington: The Science and Practice of Pharmacy, 20th Ed., A. Gennaro (ed.), Lippincott Williams & Wilkins (2000).

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)-ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

The pharmaceutical compositions may comprise pharmaceutically acceptable carriers. As used herein, “pharmaceutically acceptable carrier” refers to a material that is compatible with a recipient subject (a human) and is suitable for delivering an active agent to the target site without terminating the activity of the agent. The toxicity or adverse effects, if any, associated with the carrier preferably are commensurate with a reasonable risk/benefit ratio for the intended use of the active agent.

Pharmaceutically acceptable carriers that may be used in these compositions include ion exchangers, alumina, aluminum stearate, lecithin, serum proteins, such as human serum albumin, buffer substances such as phosphates or carbonates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions for use in the methods of the present disclosure may be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may contain stabilizers, pH modifiers, surfactants, solubilizing agents, bioavailability modifiers and combinations of these. These pharmaceutical compositions are formulated for pharmaceutical administration to a human being. Such compositions may be administered orally, parenterally, by inhalation spray, topically, rectally, nasally, buccally, vaginally or via an implanted reservoir. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. In some embodiments, the compositions are administered orally, intravenously or subcutaneously. In some embodiments, the compositions are administered orally. In some embodiments, the compositions are administered intravenously. In some embodiments, the intravenous administration can be intravenous infusion or intravenous injection. In some embodiments, the compositions are administered by an intravenous infusion. In some embodiments, the compositions are administered by subcutaneous injection. In some embodiments, the compositions are administered by intravenous infusion and then subsequently administered by subcutaneous injection. Furthermore, the compositions may be administered in a local rather than systemic means, such as administration (e.g., by injection) at a tumor site.

Pharmaceutical formulations may be prepared as liquid suspensions or solutions using a liquid, such as an oil, water, an alcohol, and combinations of these. Solubilizing agents such as cyclodextrins may be included. Pharmaceutically suitable surfactants, suspending agents, or emulsifying agents, may be added for oral or parenteral administration. Suspensions may include oils, such as peanut oil, sesame oil, cottonseed oil, corn oil and olive oil. Suspension preparations may also contain esters of fatty acids such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol; ethers, such as poly(ethyleneglycol); petroleum hydrocarbons such as mineral oil and petrolatum; and water.

Sterile injectable forms of these pharmaceutical compositions may be aqueous or oleaginous suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as sorbitan alkyl esters, such as Tweens or Spans, and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

These pharmaceutical compositions may be orally administered in any orally acceptable dosage form including capsules, tablets, aqueous suspensions or solutions. When aqueous suspensions are required for oral use, the active ingredient is combined with emulsifying and suspending agents. If desired, certain sweetening, flavoring or coloring agents may also be added. For oral administration in a capsule form, useful diluents include lactose and dried cornstarch. In the case of tablets for oral use, carriers that are commonly used include lactose and corn starch. Lubricating agents, such as magnesium stearate, are also typically added. Coatings may be used for a variety of purposes, e.g., to mask taste, to affect the site of dissolution or absorption, or to prolong drug action. Coatings may be applied to a tablet or to granulated particles for use in a capsule.

Alternatively, these pharmaceutical compositions may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

These pharmaceutical compositions may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be affected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of the present disclosure include mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active component(s) suspended or dissolved in one or more pharmaceutically acceptable carriers. Suitable carriers include mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In one embodiment, a compound of formula I-263a is formulated as a solution for intravenous infusion. In some embodiments, a compound of formula I-263a is formulated in a solution with a buffering agent or a PH modifying agent, and a cyclodextrin, such as a beta-cyclodextrin. In one embodiment, the solution includes phosphoric acid and Captisol (betadex sulfobutyl ether sodium) in water.

In some embodiments, a compound of formula I-263a is formulated as a drug product, wherein the drug product contains compound I-263a in a solution of phosphoric acid and Captisol (betadex sulfobutyl ether sodium) in water. In some embodiments, the drug product is packaged with a volume of 10 mL of compound I-263a sterile solution.

In some embodiments, the SAE inhibitor or the anti-CD19 antibody described herein may be manufactured for inclusion in a kit. A “kit” is any article of manufacture (e.g., a package or container) comprising at least one reagent or chemotherapeutic agent. A kit for use in the methods herein may comprise an SAE inhibitor, such as a compound of formula I-263a or a pharmaceutically acceptable salt thereof. In some embodiments, the kit may further include an anti-CD19 antibody, and optionally one or more additional therapeutic agents. In some embodiments, the kit may include a compound of formula I-263a or a pharmaceutically acceptable salt thereof, an anti-CD19 antibody, and optionally one or more additional therapeutic agents. In some embodiments, the kit may include one or more SAE inhibitors or pharmaceutically acceptable salts thereof. In some embodiments, the kit may include one or more anti-CD19 antibodies.

In some embodiments, the present disclosure relates to a kit comprising a medicament for use in treating cancer in a patient in need of such treatment. The kit can comprise a medicament comprising an SAE inhibitor, and optionally instructions for administering the SAE inhibitor and an anti-CD19 antibody; or the kit can comprise a medicament comprising an anti-CD19 antibody, and optionally instructions for administering the anti-CD19 antibody and an SAE inhibitor. The kit may contain a medicament comprising an SAE inhibitor and an anti-CD19 antibody, and optionally instructions for administering the SAE inhibitor and the anti-CD19 antibody, wherein the medicament is in single dosage form or in separate dosage forms. In some embodiments, the kit optionally comprises one or more additional therapeutic agents.

In some embodiments, a kit comprising an SAE inhibitor and an anti-CD19 antibody may further include another component or reagent. In some embodiments, a reagent in the kit may be a diluent for preparing the SAE inhibitor for administration. In some embodiments, a reagent in the kit may be a diluent for preparing the anti-CD19 antibody for administration. In some embodiments, a component in the kit may be a vessel for mixing the combination of the SAE inhibitor and the anti-CD19 antibody.

In another aspect, the present disclosure relates to a kit for treating cancer comprising at least one medicament comprising at least one dose of Compound I-263a or a pharmaceutically acceptable salt thereof, and at least one medicament comprising at least one dose of an anti-CD19 antibody, said kit for treating cancer further comprising dosing instructions for administering the medicaments for treatment of the patient in recognized need thereof.

In order that this present disclosure be more fully understood, the following examples are set forth. These examples are illustrative only and are not intended to limit the scope of the present disclosure in any way.

### Antibody Sequences

TABLE 1
Tafasitamab (MOR208)

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSY |
| | | VMHWVRQAPGKGLEWIGYINPYNDGTKYNEKF |
| | | QGRVTISSDKSISTAYMELSSLRSEDTAMYYC |
| | | ARGTYYYGTRVFDYWG QGTLVTVSS |
| VL | SEQ ID NO: 8 | DIVMTQSPATLSLSPGERATLSCRSSKSLQNV |
| | | NGNTYLYWFQQKPGQSPQLLIYRMSNLNSGVP |
| | | DRFSGSGSGTEFTLTISSLEPEDFAVYYCMQ |
| | | HLEYPITFGAGTKLEIK |
| Heavy chain constant | SEQ ID NO: 9 | ASTKGPSVFPLAPSSKSTSGGTAALGCLVKDY |
| domain | | FPEPVTVSWNSGALTSGVHTFPAVLQSSGLYS |
| | | LSSVVTVPSSSLGTQTYICNVNHKPSNTKVDK |
| | | KVEPKSCDKTHTCPPCPAPELLGGPDVFLFPP |
| | | KPKDTLMISRTPEVTCVVVDVSHEDPEVQFNW |
| | | YVDGVEVHNAKTKPREEQFNSTFRVVSVLTVV |
| | | HQDWLNGKEYKCKVSNKALPAPEEKTISKTKG |
| | | QPREPQVYTLPPSREEMTKNQVSLTCLVKGFY |
| | | PSDIAVEWESNGQPENNYKTTPPMLDSDGSF |
| | | FLYSKLTVDKSRWQQGNVFSCSVMHEALHNH |
| | | YTQKSLSLSPGK |
| Light chain constant | SEQ ID NO: 10 | RTVAAPSVFIFPPSDEQLKSGTASVVCLLNNF |
| domain | | YPREAKVQWKVDNALQSGNSQESVTEQDSKDS |
| | | TYSLSSTLTLSKADYEKHKVYACEVTHQGLS |
| | | SPVTKSFNRGEC |
| Full Heavy chain | SEQ ID NO: 11 | EVQLVESGGGLVKPGGSLKLSCAASGYTFTSY |
| | | VMHWVRQAPGKGLEWIGYINPYNDGTKYNEKF |
| | | QGRVTISSDKSISTAYMELSSLRSEDTAMYYC |
| | | ARGTYYYGTRVFDYWGQGTLVTVSSASTKGP |
| | | SVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPDVFLFPPKPKDTL |
| | | MISRTPEVTCVVVDVSHEDPEVQFNWYVDGV |
| | | EVHNAKTKPREEQFNSTFRVVSVLTVVHQDW |
| | | LNGKEYKCKVSNKALPAPEEKTISKTKGQPRE |
| | | PQVYTLPPSREEMTKNQVSLTCLVKGFYPSDI |
| | | AVEWESNGQPENNYKTTPPMLDSDGSFFLYS |
| | | KLTVDKSRWQQGNVFSCSVMHEALHNHYTQK |
| | | SLSLSPGK |
| Full Light chain | SEQ ID NO: 12 | DIVMTQSPATLSLSPGERATLSCRSSKSLQNV |
| | | NGNTYLYWFQQKPGQSPQLLIYRMSNLNSGV |
| | | PDRFSGSGSGTEFTLTISSLEPEDFAVYYCMQ |
| | | HLEYPITFGAGTKLEIKRTVAAPSVFIFPPSD |
| | | EQLKSGTASVVCLLNNFYPREAKVQWKVDNAL |
| | | QSGNSQESVTEQDSKDSTYSLSSTLTLSKADY |
| | | EKHKVYACEVTHQGLSSPVTKSFNRGEC |

### EXAMPLES

### Example 1: Methods In Vitro

### Cell Lines

A panel of 9 aggressive lymphoma cell lines was analyzed (7 DLBCL and 2 Burkitt lymphoma). OCI-LY10 and TMD8 cells were cultured in Iscove's modified Dulbecco medium supplemented with either 20% human plasma or 10% FCS. All other cell lines were cultured in RPMI medium (Gibco) supplemented with 10%-20% FCS (Sigma). Cells were cultured at 37° C. in 5% CO2.

### Isolation of PBMCs

Whole Blood was collected from healthy volunteers. PBMCs were isolated via density-gradient centrifugation using Biocoll (Biochrome) or Pancoll (PAN Biotech) separating solution and SepMate tubes (STEMCELL Technologies).

### ADCC Assay

PBMC effector cells were treated with 0.1 μM TAK-981 or DMSO as negative control for 17-24 hours. Target cells were stained with 1 μM carboxyfluorescein succinimidyl ester (CFSE). Subsequently, target cells were incubated with pre-treated PBMCs at an effector to target (E:T) ratio of 10:1 in the presence or absence of 1 nM tafasitamab for 2 hours at 37° C. and 5% CO2. Dead cells were stained with 1 μg/ml 4′,6-diamidino-2-phenylindole (DAPI) and flow cytometry was used to determine cytotoxicity against target cells: Cells were gated for viable (DAPI negative) and dead target cells (DAPI positive). The specific cytotoxicity was defined as the percentages of dead target cells in the treated sample substracted by the percentage of dead target cells in the control sample without antibody.

### Degranulation of NK Cells

Freshly isolated PBMCs were first pre-treated with 1 μM TAK-981 or DMSO-containing buffer for 24 hours. PBMCs with or without TAK-981 pre-treatment were co-cultured with DAUDI cells at an E:T ratio of 10:1 and incubated with 0.1 or 10 nM tafasitamab or no antibody at 37° C. and 5% CO2. After 1 hour, 2 μM Monensin was added to each well and CD107a expression was analyzed by flow cytometry after a total incubation time of 3 hours.

### Cytokine Release

PBMCs were first pre-treated with 1 μM TAK-981 or DMSO-containing buffer for 24 hours. PBMCs with or without TAK-981 pre-treatment were co-cultured with DAUDI cells at an E:T ratio of 1:1 and incubated with or without 10 nM tafasitamab at 37° C. and 5% CO2. After 24 hours, supernatants were collected and IFNγ levels were analyzed by flow cytometry using the cytometric bead array (CBA) system.

### ADCP Assay

Monocytes were positively selected from PBMCs using CD14 MicroBeads on a MACS LS column according to the manufacturer's protocol (Miltenyi Biotec). Isoltaed cells were re-suspended in RPMI medium supplemented with 10% FCS and 1× GlutaMax. Monocytes were maturated into macrophages in T-flasks for 6 to 7 days in the presence of 50 ng/ml M-CSF.

After maturation, macrophages were stained with 2 μM CFSE and detached using 5 mM EDTA and scraping. After washing with PBS, macrophages were re-suspended in medium containing either DMSO (negative control) or 1 μM TAK-981 and allowed to re-attach in 96 well culture plates for 16-24 hours. Target cells were stained using the CellTrace Violet Cell Proliferation Kit (Invitrogen) and co-cultured with pre-treated macrophages in the presence or absence of 1 nM tafasitamab for 3 hours at an E:T ratio of 2:1. The cells were detached using 0.05% Trypsin-EDTA and transferred into multi-well flow cytometry plates. Using flow cytometry the percentage of phagocytosis was evaluated, defined as the percentage of double positive cells out of all CellTrace Violet positive cells.

### Cell Viability Assay

Direct cytotoxic effects of TAK-981 and tafasitamab on cell viability were analyzed using the CellTiter Glo Kit according to the manufacturer's protocol (Promega). SU-DHL-6 cells were incubated with 100 or 500 nM TAK-981, 5 nM tafasitamab or the drug combination on white uncoated 96 F-well clear bottom plates for 24 hours at 37° C. and 5% CO2. SU-DHL-4 cells were treated with TAK-981 for 24 hours and Tafasitamab for 96 hours. The reduction of cell viability was defined as the percentage of luminescence reduction compared to medium control sample.

### Statistical Analysis

Statistical analysis was performed with GraphPad Prism8. Comparison between mean values in two groups were based on a paired two-tailed t test. *p<0.05, **p<0.01, ***p<0.001, n.s. not significant.

### Results (In Vitro)

For in vitro analysis ADCC, ADCP and direct cytotoxicity assays were performed.

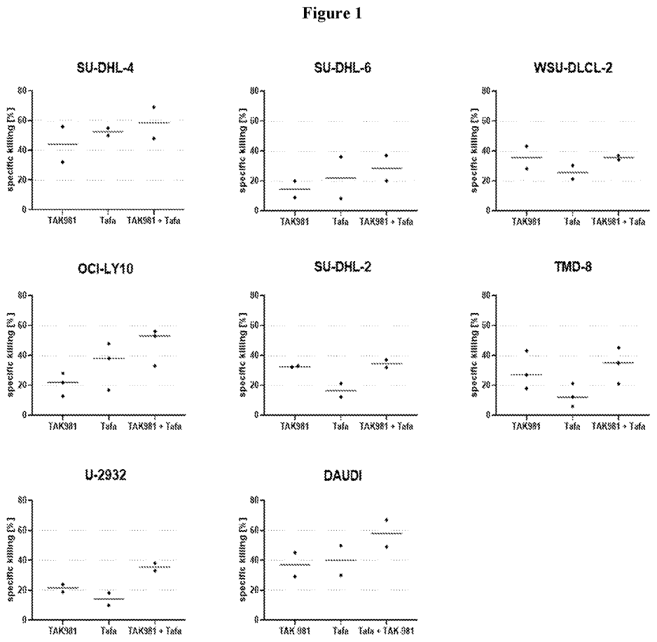

For ADCC assays, PBMCs were first pre-treated with TAK-981 and then co-incubated with target cells and tafasitamab. Both TAK-981 as well as tafasitamab single agent treatments resulted in specific killing of the different lymphoma cell lines to various extents (FIG. 1). The combination of TAK-981 and tafasitamab increased cytotoxicity compared to the respective mono treatments in 6/8 tested lymphoma cell lines (FIG. 1: SU-DHL-4, SU-DHL-6, OCI-LY10, TMD8, U-2932, DAUDI).

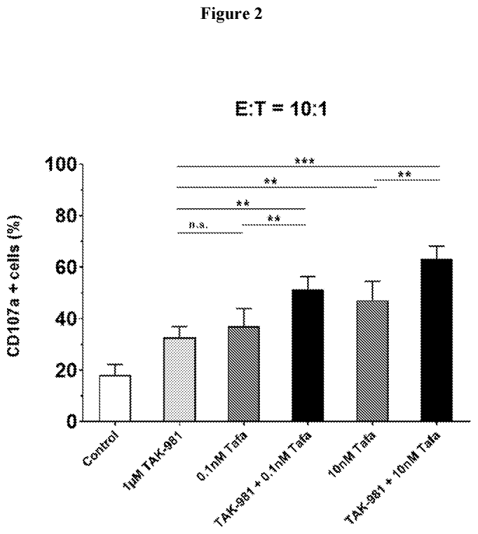

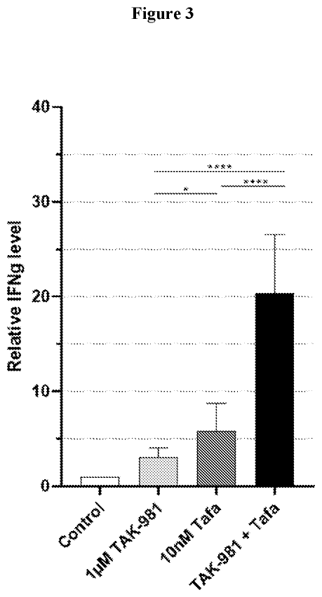

Moreover, degranulation of NK cells was determined via detection of CD107a after incubation of TAK-981- or DMSO-pre-treated PBMCs with DAUDI cells and tafasitamab or medium control. TAK-981 as well as tafasitamab treatment at two different concentrations (0.1 and 10 nM) resulted in an elevated percentage of CD107+ NK cells compared to DMSO-treated control cells (FIG. 2). Again, the combination of TAK-981 and tafasitamab resulted in a significant increase of CD107+ cells compared to the respective mono treatments. In addition, cytokine release was investigated via flow cytometry and the CBA system. TAK-981 or DMSO pre-treated PBMCs were co-incubated with DAUDI cells and tafasitamab or without antibody as negative control. Here, significantly increased IFNγ levels were detected in the supernatants of samples treated with both TAK-981 and tafasitamab in comparison to the mono treatments (FIG. 3).

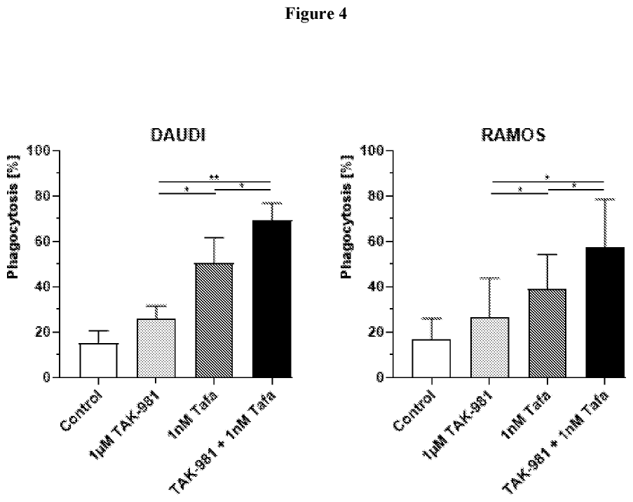

For ADCP assays, matured macrophages were pre-treated with TAK-981 and co-incubated with target cells and tafasitamab. Both cell lines (DAUDI and RAMOS) were effectively phagocytosed upon TAK-981 or tafasitamab single agent treatment (FIG. 4). The combination of TAK-981 and tafasitamab resulted in significantly enhanced phagocytosis in comparison to the respective mono treatments in both cell lines.

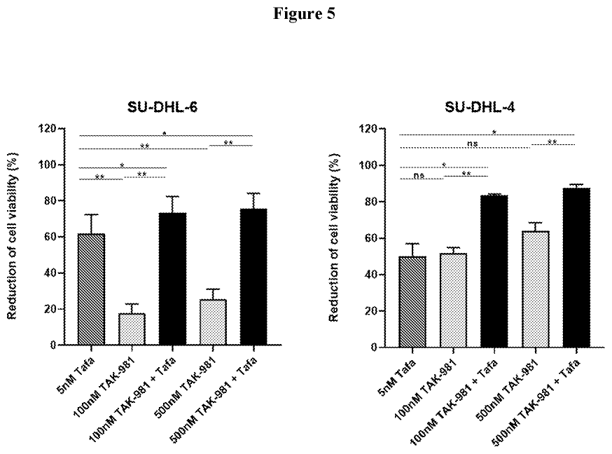

To analyze direct cytotoxic effects of the two drugs, cell viability assays quantifying ATP levels via a luminescence-based method were performed. Similar to the other tested mode of actions, the combination of both drugs significantly increased direct cytotoxic effects against SUDHL-6 and SU-DHL-4 cells compared to the single agent treatments (FIG. 5).

### Example 2: Methods (In Vivo)

### Statistical Analysis of Treatment and Combination Effect for Tumor Growth in Subcutaneous Xenograft Models

The following statistical analysis methods were utilized in each of Studies 1-3.

### Growth Rate Inhibition:

The differences in the tumor growth trends over time between pairs of treatment groups were assessed by fitting each animal's data to a simple exponential growth model and comparing the mean growth rates of the two groups, as described in more detail below. The difference in the growth rates was summarized by the GRI, which is the reduction in growth rate experienced by the treatment group relative to that of the reference group, expressed as a fraction of the vehicle growth rate.

$$Growth rate inhibition = \frac{{μ}_{C} - {μ}_{T}}{{μ}_{V}} \times 100 \%$$

μ_{T} and μ_{C} are the mean tumor growth rates for the treatment and reference groups, respectively. μ_{V} is the mean tumor growth rate for the vehicle group, which in most cases is the same as the reference group.

A positive GRI indicates that the tumors in the treatment group grew at a reduced rate relative to the reference group. A GRI greater than 100% is interpreted as tumor regression when the vehicle group is the same as the reference group. A negative value of the GRI means that the tumors in the treatment group grew faster than the tumors in the reference group. Statistical significance was determined using an unpaired t-test. A statistically significant P value suggests that the trends over time for the two treatment groups were different.

### Tumor Growth Rate Computation:

Low tumor volumes can cause problems with the data analysis, so all measurements below 25 cubic mm (including 0 values) were excluded from the analysis. After this exclusion step, the tumor volume was assumed to follow an exponential growth model. More specifically, for a given animal and treatment group,

$${log}_{10} ( {V}_{i} ) = a + b \times {t}_{i} + {ε}_{i}$$

where Vdi is the tumor volume at the i^{th} time point. Here, a is the initial log volume, b is the tumor growth rate, and tᵢ is the measurement time in days. εᵢ is the residual error term, which was assumed to be uncorrelated and drawn from a normal distribution.

This model was fit separately for each animal within each treatment group. If an animal was sacrificed or died early, but at least two unique time points were measured (including baseline), then the data up to that point was used to estimate the growth rate. If the animal had measurements at less than two time points, then the animal was automatically excluded from the analysis.

In rare cases, the estimated growth rate for one or more animals might be very different from the other animals within the same group. To make the analysis robust, an interval was defined with a width of 30 times the median absolute deviation of the estimated growth rates for a given group. The interval was centered at the median of the growth rates for the group. If the growth rate for any animal fell outside this interval, the growth rate was replaced with the value at the boundary of the interval.

### Combination Analysis

Combination analysis was performed to determine if there was a benefit from combining drug treatments. This analysis was also based on the estimated tumor growth rates. The measure of synergy was defined as

$$Synergy score = \frac{{μ}_{AB} - {μ}_{A} - {μ}_{B} + {μ}_{Control}}{{μ}_{V}} \times 100 \%$$

Here, μ_{AB}, μ_{A}, μ_{B}, and μ_{Control} are the mean growth rates for the combination, drug A, drug B, and control groups, respectively. As before, μ_{V} is the mean tumor growth rate for the vehicle group, which in most cases is the same as the control group. The standard error of the synergy score was calculated as the square root of the sum of squared standard errors across the four groups. The degrees of freedom were estimated using the Welch-Satterthwaite equation. A hypothesis test was performed to determine if the synergy score differed from 0. P-values were calculated by dividing the synergy score by its standard error and tested against a t-distribution (two-tailed) with the above-calculated degrees of freedom. A P value of less than 0.05 is statistically significant.

The combination results can be interpreted as follows. Statistically significant negative synergy scores indicate a synergistic combination (“Syn.”). Statistically significant positive synergy scores indicate a sub-additive combination (“Sub-add.”) when the combination performs better (i.e. has a lower growth rate) than the best performing single agent. Statistically significant positive synergy scores indicate an antagonistic combination (“Antag.”) when the combination performs worse than the best performing single agent. Scores that are not statistically significant should be considered additive (“Add.”).

### Study 1:

Daudi is a human Burkitt's Lymphoma cell line. A Daudi human xenograft tumor model was generated by subcutaneous inoculation in the flank of 9 week old female CB17 SCID mice (Charles River Laboratories, Wilmington, MA) with 2.0×10⁶ Daudi cells (cell suspension in 0.1 ml RPMI with Matrigel 1:1). When the mean tumor volume reached approximately 140 mm³, the animals were randomized into one vehicle control group and seven treatment groups (n=7/group) as described in Table 1a. Mice were then dosed with 20% HPβCD or Compound I-263a and/or tafasitamab over a 3 week period. In the combination treatment groups, I-263a was administered first, followed immediately by the administration of tafasitamab. Tumor growth and body weight were measured twice per week during the treatment and post-treatment periods, and mice were humanely euthanized once they had reached their humane endpoint. Tumors were measured using Vernier calipers and volumes were calculated using standard equation: V=W²×L/2, where V=volume, W=width, and L=length for the tumor.

A 0.8625 mg/mL stock solution of I-263a was formulated weekly in 20% HPβCD and administered intravenously (IV) based on an average body weight using a 0.2 mL dosing volume. Final dose of I-263a received was 7.5 mg/kg. I-263a was administered on a BIW schedule for 3 weeks (Days 0, 4, 7, 11, 14, 18). Tafasitamab was formulated prior to each injection in 0.9% saline at 0.354, 1.15, and 2.3 mg/mL. Final dose of tafasitamab received was 3, 10, or 20 mg/kg based on average body weight. Tafasitamab was administered intraperitoneally (IP) using a 0.2 mL dosing volume, on a BIW schedule for 3 weeks (Days 0, 4, 7, 11, 14, 18). The vehicle group received IV treatment with the vehicle for I-263a (20% HPβCD) on a BIW schedule for three weeks (Days 0, 4, 7, 11, 14, 18).

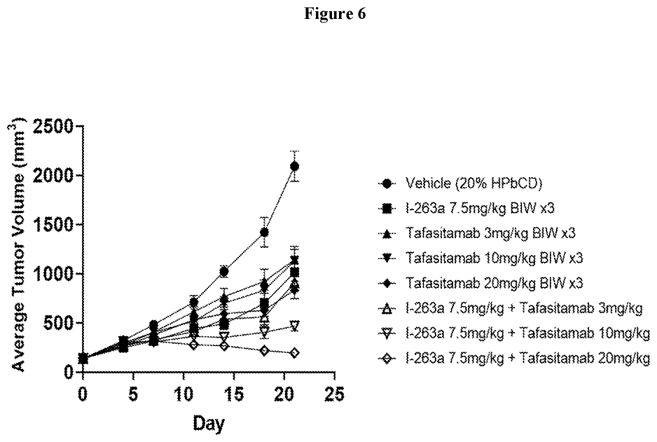

Statistical analysis for growth rate inhibition was performed on data up to and including Day 21. Average tumor growth curves through day 21 are shown in FIG. 6.

In pairwise comparisons with vehicle, all treatments resulted in tumor growth rate inhibition relative to vehicle treated tumors on Day 21 of the study (p<0.01 for all) as shown in Table 1a. Using the values for growth rate inhibition on Day 21, the combination of I-263a at 7.5 mg/kg and tafasitamab at 20 mg/kg was scored as synergistic, while the combination of I-263a at 7.5 mg/kg and tafasitamab at 3 mg/kg or 10 mg/kg was scored as additive (Table 1b). These results demonstrate that I-263a as a single agent inhibits tumor growth in a Daudi human Burkitt's lymphoma mouse model, as does tafasitamab as a single agent. When I-263a and 20 mg/kg tafasitamab are combined, the two agents show synergistic antitumor activity on Day 21 with almost complete control of tumor growth compared to the initial volume (GRI of 98%).

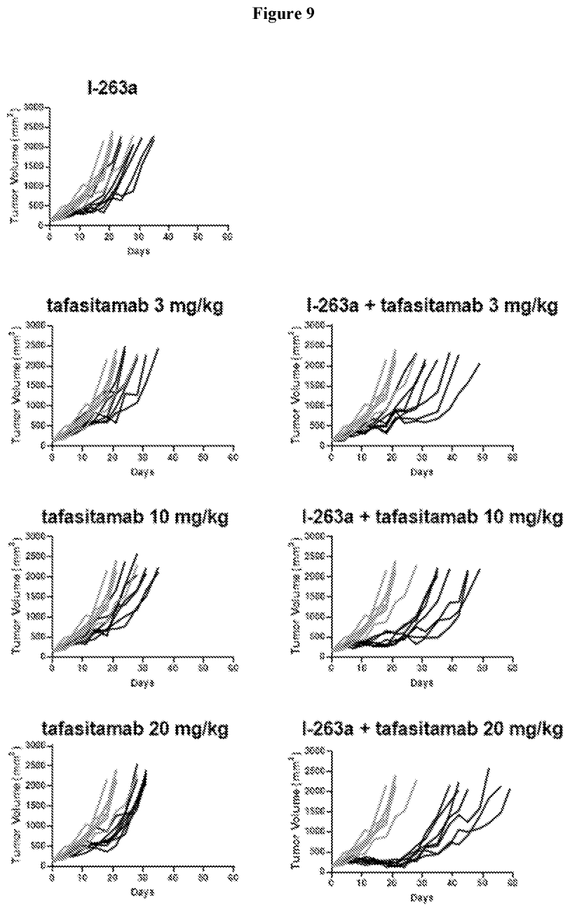

Combination benefit of I-263a with tafasitamab in the Daudi model is further evidenced by the tumor growth kinetics during the post treatment period (see FIG. 9: gray lines represent the vehicle groups and black lines represent the treatment groups). In the vehicle and single agent groups, all mice were removed from study by Day 35 due to reaching the tumor volume associated with humane endpoint, whereas some mice in the combination groups remained on study beyond Day 35 (n=3, 4, and 7 mice in the combination groups with 3, 10, and 20 mg/kg tafasitamab, respectively).

TABLE 1a
Combination of Compound I-263a and tafasitamab in the Daudi xenograft model

| | | | | Tumor | | |
|---|---|---|---|---|---|---|
| Study | | Dosing | | volume on | | |
| Group | Treatment | Regimen | Route | Day 21 (±SEM) | GRI % | P-value |
| A | 20% HPβCD | BIW | IV | 2091.4 ± 153.8 | NA | NA |
| B | I-263a 7.5 mg/kg | BIW | IV | 1015.9 ± 137.9 | 32 | 0.004 |
| C | Tafasitamab 3 mg/kg | BIW | IP | 1142 ± 135.1 | 24 | 0.006 |
| D | Tafasitamab 10 mg/kg | BIW | IP | 1132.7 ± 116.5 | 28 | <0.001 |
| E | Tafasitamab 20 mg/kg | BIW | IP | 835.2 ± 89.1 | 38 | <0.001 |
| F | I-263a 7.5 mg/kg + | BIW | IV/IP | 910.4 ± 88.2 | 38 | <0.001 |
| | Tafasitamab 3 mg/kg | | | | | |
| G | I-263a 7.5 mg/kg + | BIW | IV/IP | 468.1 ± 48.7 | 66 | <0.001 |
| | Tafasitamab 10 mg/kg | | | | | |
| H | I-263a 7.5 mg/kg + | BIW | IV/IP | 196.2 ± 24.9 | 98 | <0.001 |
| | Tafasitamab 20 mg/kg | | | | | |

TABLE 1b
Classification for in vivo combination of Compound I-263a
and tafasitamab in the Daudi xenograft model: Day 21

| | Synergy | | | |
|---|---|---|---|---|
| Treatment | score | SEM | P-value | Classification |
| I-263a 7.5 mg/kg + | 18 | 12 | 0.137 | Additive |
| Tafasitamab 3 mg/kg | | | | |
| I-263a 7.5 mg/kg + | −6 | 11 | 0.6 | Additive |
| Tafasitamab 10 mg/kg | | | | |
| I-263a 7.5 mg/kg + | −28 | 11 | 0.021 | Synergistic |
| Tafasitamab 20 mg/kg | | | | |

### Study 2:

OCI-Ly10 is a human DLBCL cell line. An OCI-Ly10 human xenograft tumor model was generated by subcutaneous inoculation in the flank of 8 week old female CB17 SCID mice (Charles River Laboratories, Wilmington, MA) with 4.0×10⁶ OCI-Ly10 cells (cell suspension in 0.1 ml IMDM with Matrigel 1:1). When the mean tumor volume reached approximately 175 mm³, the animals were randomized into one vehicle control group and 7 treatment groups (n=7/group) as described in Table 2a. Mice were then dosed with 20% HPβCD or Compound I-263a and/or tafasitamab over a 3 week period. In the combination treatment groups, I-263a was administered first, followed immediately by the administration of tafasitamab. Tumor growth and body weight were measured twice per week during the treatment and post-treatment periods, and mice were humanely euthanized once they had reached their humane endpoint. Tumors were measured using Vernier calipers and volumes were calculated using standard equation: V=W²×L/2, where V=volume, W=width, and L=length for the tumor.

A 0.75 mg/mL stock solution of I-263a was formulated weekly in 20% HPβCD and administered intravenously (IV) based on an average body weight using a 0.2 mL dosing volume. Final dose of I-263a received was 7.5 mg/kg. I-263a was administered on a BIW schedule for 3 weeks (Days 0, 4, 7, 11, 14, 18). Tafasitamab was formulated prior to each injection in 0.9% saline at 0.3, 1.0, and 2.0 mg/mL. Final dose of tafasitamab received was 3, 10, or 20 mg/kg based on average body weight. Tafasitamab was administered intraperitoneally (IP) using a 0.2 mL dosing volume, on a BIW schedule for 3 weeks (Days 0, 4, 7, 11, 14, 18). The vehicle group received IV treatment with the vehicle for I-263a (20% HPβCD) on a BIW schedule for three weeks (Days 0, 4, 7, 11, 14, 18).

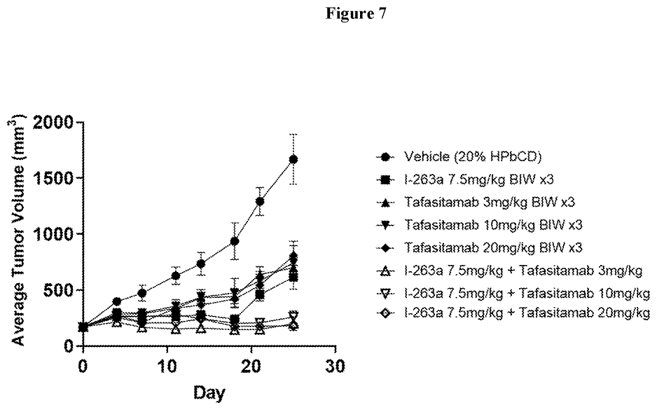

Statistical analysis for growth rate inhibition was performed on data up to and including Day 25. Average tumor growth curves through day 25 are shown in FIG. 7.

In pairwise comparisons with vehicle, all treatments resulted in tumor growth rate inhibition relative to vehicle treated tumors on Day 25 of the study (p<0.01 for all) as shown in Table 2a. Using the values for growth rate inhibition on Day 25, the combination of I-263a at 7.5 mg/kg and tafasitamab at 3, 10, or 20 mg/kg was scored as additive (Table 2b). These results demonstrate that I-263a as a single agent inhibits tumor growth in a human OCI-Ly10 DLBCL mouse model, as does tafasitamab as a single agent. When I-263a and tafasitamab are combined, the two agents show additive antitumor activity on Day 25, with almost complete prevention of tumor growth above the initial volume (GRI of 102-112%).

TABLE 2a
Combination of Compound I-263a and tafasitamab
in the OCI-Ly10 xenograft model

| | | | | Tumor | | |
|---|---|---|---|---|---|---|
| Study | | Dosing | | volume on | | |
| Group | Treatment | Regimen | Route | Day 25 (±SEM) | GRI % | P-value |
| A | 20% HPβCD | BIW | IV | 1668.8 ± 223.8 | NA | |
| B | I-263a 7.5 mg/kg | BIW | IV | 615.9 ± 106.4 | 55 | <0.001 |
| C | Tafasitamab 3 mg/kg | BIW | IP | 701 ± 94.2 | 37 | 0.005 |
| D | Tafasitamab 10 mg/kg | BIW | IP | 745.1 ± 154.2 | 43 | 0.004 |
| E | Tafasitamab 20 mg/kg | BIW | IP | 809.2 ± 129.1 | 36 | 0.01 |
| F | I-263a 7.5 mg/kg + | BIW | IV/IP | 202.5 ± 61 | 112 | <0.001 |
| | Tafasitamab 3 mg/kg | | | | | |
| G | I-263a 7.5 mg/kg + | BIW | IV/IP | 260.7 ± 51.4 | 102 | <0.001 |
| | Tafasitamab 10 F | | | | | |
| H | I-263a 7.5 mg/kg + | BIW | IV/IP | 182 ± 19.6 | 109 | <0.001 |
| | Tafasitamab 20 mg/kg | | | | | |

TABLE 2b
Classification for in vivo combination of Compound I-263a
and tafasitamab in the OCI-Ly10 xenograft model: Day 25

| | Synergy | | | |
|---|---|---|---|---|
| Treatment | score | SEM | P-value | Classification |
| I-263a 7.5 mg/kg + | −21 | 17 | 0.241 | Additive |
| Tafasitamab 3 mg/kg | | | | |
| I-263a 7.5 mg/kg + | −4 | 16 | 0.792 | Additive |
| Tafasitamab 10 mg/kg | | | | |
| I-263a 7.5 mg/kg + | −18 | 14 | 0.213 | Additive |
| Tafasitamab 20 mg/kg | | | | |

### Study 3:

WSU-DLCL2 is a human DLBCL cell line. A WSU-DLCL2 human xenograft tumor model was generated by subcutaneous inoculation in the flank of 8 week old female CB17 SCID mice (Charles River Laboratories, Wilmington, MA) with 4.0×10⁶ WSU-DLCL2 cells (cell suspension in 0.1 mL RPMI 1640 media). When the mean tumor volume reached approximately 200 mm³, the animals were randomized into one vehicle control group and seven treatment groups (n=7/group) as described in Table 3a. Mice were then dosed with 20% HPβCD or Compound I-263a and/or tafasitamab over a 3 week period. In the combination treatment groups, I-263a was administered first, followed immediately by the administration of tafasitamab. Tumor growth and body weight were measured twice per week during the treatment and post-treatment periods, and mice were humanely euthanized once they had reached their humane endpoint. Tumors were measured using Vernier calipers and volumes were calculated using standard equation: V=W²×L/2, where V=volume, W=width, and L=length for the tumor.

A 0.8625 mg/mL stock solution of I-263a was formulated weekly in 20% HPβCD and administered intravenously (IV) based on an average body weight using a 0.2 mL dosing volume. Final dose of I-263a received was 7.5 mg/kg. I-263a was administered on a BIW schedule for 3 weeks (Days 1, 4, 8, 11, 15, 18). Tafasitamab was formulated prior to each injection in 0.9% saline at 0.6 mg/mL and 2.0 mg/mL. Final dose of tafasitamab received was 3, 10, or 20 mg/kg based on average body weight. Tafasitamab was administered intraperitoneally (IP) on a BIW schedule for 3 weeks (Days 1, 4, 8, 11, 15, 18). For the 3 mg/kg dose level, 0.1 mL of the 0.6 mg/mL solution was administered. For the 10 mg/kg and 20 mg/kg dose levels, dosing volumes of the 2.0 mg/mL solution were 0.1 mL and 0.2 mL, respectively. The vehicle group received IV treatment with the vehicle for I-263a (20% HPβCD) on a BIW schedule for three weeks (Days 1, 4, 8, 11, 15, 18).

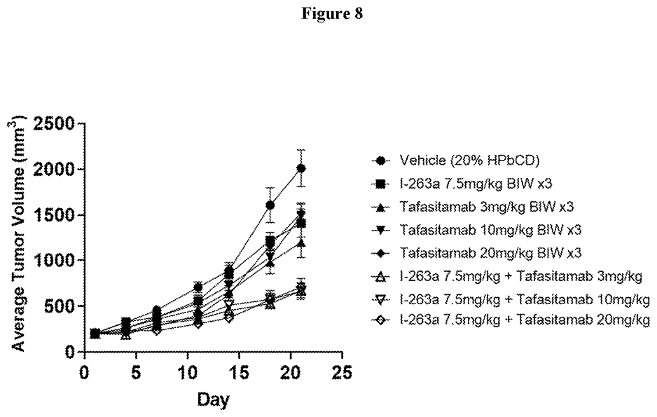

Statistical analysis for growth rate inhibition was performed on data up to and including Day 21. Average tumor growth curves through day 21 are shown in FIG. 8.

In pairwise comparisons with vehicle, single agent treatment with TAK-981 at 7.5 mg/kg or with tafasitamab at 10 mg/kg or 20 mg/kg did not result in significant growth rate inhibition relative to vehicle treated tumors on Day 21 of the study (p>0.01 for all) as shown in Table 3a. Tafasitamab at 3 mg/kg resulted in a statistically significant but minor degree of growth inhibition (20% GRI, p=0.019). The combination of TAK-981 with tafasitamab at 3, 10, or 20 mg/kg resulted in significant growth rate inhibition of 44, 46, and 46% respectively (p<0.001 for all). Using the values for growth rate inhibition on Day 21, the combination of I-263a at 7.5 mg/kg and tafasitamab at 20 mg/kg was scored as synergistic, while the combination of I-263a at 7.5 mg/kg and tafasitamab at 3 mg/kg or 10 mg/kg was scored as additive (Table 3b). These results demonstrate that I-263a and tafasitamab as single agents do not show significant or extensive tumor growth inhibition in a WSU-DLCL2 human DLBCL mouse model. When I-263a and tafasitamab are combined, significant GRI is observed, with the 20 mg/kg dose level of tafasitamab resulting in synergistic antitumor activity with TAK-981.

TABLE 3a
Combination of Compound I-263a and tafasitamab
in the WSU-DLCL2 xenograft model

| | | | | Tumor | | |
|---|---|---|---|---|---|---|
| Study | | Dosing | | volume on | | |
| Group | Treatment | Regimen | Route | Day 21 (±SEM) | GRI % | P-value |
| A | 20% HPβCD | BIW | IV | 2010.5 ± 201.0 | | |
| B | I-263a 7.5 mg/kg | BIW | IV | 1410.7 ± 155.8 | 14 | 0.099 |
| C | Tafasitamab 3 mg/kg | BIW | IP | 1197.3 ± 168.3 | 20 | 0.019 |
| D | Tafasitamab 10 mg/kg | BIW | IP | 1494.3 ± 126.3 | 12 | 0.149 |
| E | Tafasitamab 20 mg/kg | BIW | IP | 1519.0 ± 110.8 | 2 | 0.862 |
| F | I-263a 7.5 mg/kg + | BIW | IV/IP | 667.8 ± 77.9 | 44 | <0.001 |
| | Tafasitamab 3 mg/kg | | | | | |
| G | I-263a 7.5 mg/kg + | BIW | IV/IP | 663.9 ± 93.6 | 46 | <0.001 |
| | Tafasitamab 10 mg/kg | | | | | |
| H | I-263a 7.5 mg/kg + | BIW | IV/IP | 709.5 ± 93.2 | 46 | 0.001 |
| | Tafasitamab 20 mg/kg | | | | | |

TABLE 3b
Classification for in vivo combination of Compound I-263a
and tafasitamab in the WSU-DLCL2 xenograft model: Day 21

| | Synergy | | | |
|---|---|---|---|---|
| Treatment | score | SEM | P-value | Classification |
| I-263a 7.5 mg/kg + | −10 | 10 | 0.312 | Additive |
| Tafasitamab 3 mg/kg | | | | |
| I-263a 7.5 mg/kg + | −20 | 12 | 0.099 | Additive |
| Tafasitamab 10 mg/kg | | | | |
| I-263a 7.5 mg/kg + | −30 | 13 | 0.03 | Synergistic |
| Tafasitamab 20 mg/kg | | | | |

What is claimed is:

1. A method of treating a cancer, comprising administering to a human subject in need of said treatment a combination of an SAE inhibitor or a pharmaceutically acceptable salt thereof, and an anti-CD19 antibody.

2. The method of claim 1, wherein the SAE inhibitor is [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a).

3. The method of any one of claims 1-2, wherein the anti-CD19 antibody comprises a heavy chain variable region comprising an HCDR1 region comprising the sequence SYVMH (SEQ ID NO: 1), an HCDR2 region comprising the sequence NPYNDG (SEQ ID NO: 2), and an HCDR3 region comprising the sequence GTYYYGTRVFDY (SEQ ID NO: 3) and a light chain variable region comprising the sequence LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

4. The method of any one of claims 1-3, wherein the anti-CD19 antibody comprises a heavy chain variable region of EVOLVESGGGLVKPGGSLKLSCAASGYTFTSYVMHWVRQAPGKGLEWIGYINPY NDGTKYNEKFQGRVTISSDKSISTAYMELSSLRSEDTAMYYCARGTYYYGTRVF DYWGQGTLVTVSS (SEQ ID NO: 7) and a light chain variable region of DIVMTQSPATLSLSPGERATLSCRSSKSLQNVNGNTYLYWFQQKPGQSPQLLIYR MSNLNSGVPDRFSGSGSGTEFTLTISSLEPEDFAVYYCMQHLEYPITFGAGTKLEI K (SEQ ID NO: 8).

5. The method of any one of claims 1-5, wherein the anti-CD19 antibody comprises an Fc domain comprising an amino acid substitution at position S239 and/or 1332, wherein the numbering is according to the EU index as in Kabat.

6. The method of any one of claims 1-4, wherein the anti-CD19 antibody comprises an Fc domain comprising an S239D amino acid substitution and an 1332E amino acid substitution, wherein the numbering is according to the EU index as in Kabat.

7. The method of any one of claims 1-6, wherein the anti-CD19 antibody comprises a heavy chain region of EVOLVESGGGLVKPGGSLKLSCAASGYTFTSYVMHWVRQAPGKGLEWIGYINPY NDGTKYNEKFQGRVTISSDKSISTAYMELSSLRSEDTAMYYCARGTYYYGTRVF DYWGQGTLVTVSSASTKGPSVFPLAPSSKSTSGGTAALGCLVKDYFPEPVTVSW NSGALTSGVHTFPAVLQSSGLYSLSSVVTVPSSSLGTQTYICNVNHKPSNTKVDK KVEPKSCDKTHTCPPCPAPELLGGPDVFLFPPKPKDTLMISRTPEVTCVVVDVSHE DPEVQFNWYVDGVEVHNAKTKPREEQFNSTFRVVSVLTVVHQDWLNGKEYKC KVSNKALPAPEEKTISKTKGQPREPQVYTLPPSREEMTKNQVSLTCLVKGFYPSDI AVEWESNGQPENNYKTTPPMLDSDGSFFLYSKLTVDKSRWQQGNVFSCSVMHE ALHNHYTQKSLSLSPGK (SEQ ID NO: 11) and a light chain region of DIVMTQSPATLSLSPGERATLSCRSSKSLQNVNGNTYLYWFQQKPGQSPQLLIYR MSNLNSGVPDRFSGSGSGTEFTLTISSLEPEDFAVYYCMQHLEYPITFGAGTKLEI KRTVAAPSVFIFPPSDEQLKSGTASVVCLLNNFYPREAKVQWKVDNALQSGNSQ ESVTEQDSKDSTYSLSSTLTLSKADYEKHKVYACEVTHQGLSSPVTKSFNRGEC (SEQ ID NO: 12)

8. The method of any one of claims 1-7, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered orally.

9. The method of any one of claims 1-8, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered intravenously, or subcutaneously.

10. The method of any one of claims 1-9, wherein the [(1R,2S,4R)-4-{[5-({4-[(1R)-7-chloro-1,2,3,4-tetrahydroisoquinolin-1-yl]-5-methyl-2-thienyl}carbonyl)pyrimidin-4-yl]amino}-2-hydroxycyclopentyl]methyl sulfamate (Compound I-263a) or a pharmaceutically acceptable salt thereof, is administered by intravenous infusion.

11. The method of any one of claims 1-10, wherein the cancer is a CD19 positive cancer.

12. The method of any one of claims 1-11, wherein the cancer is a hematological malignancy.

13. The method of any one of claims 1-12, wherein the cancer is a lymphoma or a leukemia.

14. The method of any one of claims 1-13, wherein the cancer is chronic lymphocytic leukemia, acute lymphoblastic leukemia, or a non-Hodgkin's lymphoma.

15. The method of any one of claims 1-14, wherein the cancer is a non-Hodgkin's lymphoma selected from the group consisting of follicular lymphoma (FL), small lymphocytic lymphoma, mucosa-associated lymphoid tissue lymphoma, marginal zone lymphoma (MZL), mantle cell lymphoma (MCL), diffuse large B-cell lymphoma (DLBCL), and Burkitt lymphoma.


SEQUENCE LISTING (13 sequences)

SEQ ID NO: 1 - protein, 5 aa, synthetic construct
  source 1..5: note = HCDR1
SYVMH

SEQ ID NO: 2 - protein, 6 aa, synthetic construct
  source 1..6: note = HCDR2
NPYNDG

SEQ ID NO: 3 - protein, 12 aa, synthetic construct
  source 1..12: note = HCDR3
GTYYYGTRVFDY

SEQ ID NO: 4 - protein, 16 aa, synthetic construct
  source 1..16: note = LCDR1
RSSKSLQNVNGNTYLY

SEQ ID NO: 5 - protein, 7 aa, synthetic construct
  source 1..7: note = LCDR2
RMSNLNS

SEQ ID NO: 6 - protein, 9 aa, synthetic construct
  source 1..9: note = LCDR3
MQHLEYPIT

SEQ ID NO: 7 - protein, 121 aa, synthetic construct
  source 1..121: note = VH
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, synthetic construct
  source 1..112: note = VL
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, synthetic construct
  source 1..330: note = Heavy chain constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 451 aa, synthetic construct
  source 1..451: note = Full Heavy chain
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, synthetic construct
  source 1..219: note = Full Light chain
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]